(19) United States
(12) Patent Application Publication
(10) Pub. No.: US 2018/0116946 A1
(43) Pub. Date: May 3, 2018

(54) NOVEL COSMETIC EMULSIONS PROVIDED IN FOAM FORM, METHOD FOR OBTAINING SAME AND COSMETIC USE THEREOF

(71) Applicant: SOCIETE D'EXPLOITATION DE PRODUITS POUR LES INDUSTRIES CHIMIQUES SEPPIC, Paris Cedex 07 (FR)

(72) Inventors: Cecile TAILLEBOIS, Salies (FR); Severine SIGURANI, Toulouse (FR); Benoit SOUYRI, Castres (FR)

(21) Appl. No.: 15/567,835

(22) PCT Filed: Apr. 8, 2016
(86) PCT No.: PCT/FR2016/050820

§ 371 Date: Oct. 19, 2017

(30) Foreign Application Priority Data
Apr. 20, 2015 (FR) 1553511
Nov. 17, 2015 (FR) 1561066

(51) Int. Cl. A61K 8/86; A61K 8/06; A61K 8/04; A61K 8/25; A61K 8/73; A61Q 5/02; A61Q 19/10; A61Q 1/00

(52) U.S. Cl. CPC A61K 8/86; A61K 8/062; A61K 8/046; A61K 8/25; A61K 2800/48; A61Q 5/02; A61Q 19/10; A61Q 1/00; A61K 8/73

(57) ABSTRACT

Disclosed is a foam, oil-in-water emulsion including, for 100% of the weight thereof, a) 27-89.9 wt. % aqueous phase (A₁), b) 0.1-3 wt. % cross-linked anionic polyelectrolyte (P) from the polymerization, in the presence of at least one cross-linking agent, of partially or totally salified 2-methyl-2-[(1-oxo-2-propenyl)amino]-1-propanesulfonic acid, with at least one neutral monomer selected among acrylamide, (2-hydroxyethyl) acrylate, N,N-dialkyl acrylamides, in which each of the alkyl groups includes 1-4 carbon atoms, and at least one monomer of formula (I): R=radical C₈-C₂₀ and 1≤n≤20, c) 5-20 wt. % of at least one inorganic thickening agent (Ei), and d) 5-50 wt. % of an oil phase (A₂) including, for 100% of the weight thereof: d₁) 0.2-25 wt. % emulsifying system (S), d₂) 75-99.8 wt. % of at least one oil and optionally of at least one wax. 6/1≤(Ei)/(P)≤20/1. Methods of preparation and use are also disclosed.

The subject of the present invention is novel emulsions in the form of foams, the preparation process thereof, the use thereof as topical cosmetic compositions intended for caring for and cleansing the skin, hair, scalp, mucous membranes and nails, and also for making up the skin, nails and hair.

The cosmetic compositions may be in a single-phase form, such as, for example, aqueous or aqueous-alcoholic or aqueous-glycolic solutions, in which all the constituents of the cosmetic composition are soluble in a solvent. However, the most commonly used cosmetic compositions are in a multi-phase form and more particularly in what is referred to as a “dispersed” form, for which a liquid or gas phase is kept dispersed in a stable manner in a continuous liquid phase.

Among the dispersed forms commonly used by the cosmetics and pharmaceutical industries, mention may be made of compositions in the form of foams.

Foams are structures consisting of a set of gaseous spherical or polyhedral cells separated by thin lamellae of liquids formed by the juxtaposition of bubbles generated by a gas dispersed in a liquid. If the lamellae are disrupted or if they flow back into the gaseous cells, coalescence of the gaseous cells is then observed which results in destabilization of the overall structure and ultimately in the separation of the liquid phase and the gas phase and thus in the disappearance of the foam form. Foams are also thermodynamically unstable galenical forms and the challenge of the researcher is then to find means of reinforcing the stability of the foam in order to avoid too rapid a disappearance of this physical form. Thus, a composition in foam physical form may be stabilized over time by the use of a suitable surfactant system and/or an auxiliary chemical agent selected from elements included in the group consisting of fatty amines, fatty alcohols, fatty alkanolamides or tertiary amine oxides, as described in the international patent application published under the number WO 93/22538 A1.

Foams are generally prepared from liquid compositions by mixing a gas, such as, for example, air or nitrogen or carbon dioxide, provided that the liquid in which the gas is dispersed comprises at least one suitable surfactant, which has both properties of interfacial activity and the ability to organize the liquid at the interface in the form of a film. The mixing of the gas in said liquid compositions comprising a suitable surfactant intended to achieve the form of a foam is generally carried out by bubbling said gas but can also be carried out by very high-speed stirring of said compositions under an atmosphere of the gas which is sought to be dispersed. The preferred process for forming cosmetic compositions in the form of a foam is that which introduces the gas by bubbling because the other processes require a very large supply of energy, or even the use of an unconventional stirring rotor which is highly expensive on the industrial scale.

Such cosmetic compositions comprising suitable surfactants and in foam form are characterized by a large internal specific surface area inducing a high capacity for adsorption, thereby rendering such compositions in foam form particularly suitable for a use in cleansing the skin, hair or scalp. Cosmetic compositions in the form of a foam also have the advantage of enabling a fine distribution of the active ingredients included in said foam on the skin.

A disadvantage of cosmetic compositions in foam form is the use of a large amount of surfactants, often greater than a proportion by weight of 5%, which then increases the risk of a lower skin tolerance for consumers. However, the major disadvantage of cosmetic compositions in foam form consists of the low stability of such systems, which lose their foam appearance within the 24 hours following their formation, which constitutes a stability duration that is incompatible with the requirements for stability durations of cosmetic compositions.

This disadvantage is circumvented by supplying the cosmetic composition to the consumer in liquid form in a container in which a pressurized liquefied gas serving as propellant gas is also present. By opening a pressure valve, the mixture of liquid composition and propellant gas escapes from the container through a nozzle and the evaporating propellant leaves a foam. The consumer can thus generate the foam form of the cosmetic composition before it is applied to the skin.

However, this solution commonly used by consumers relates essentially to cosmetic compositions based on suitable surfactants, which relies on a simple formulation scheme based on a few ingredients, which does not make it possible to achieve compositions in foam form which are more complex but suited to the current needs and requirements of consumers.

With this in mind, solutions intended to present emulsions in the form of foams have been developed in the prior art. Emulsions are the galenical forms most commonly used by the cosmetics industry and the pharmaceutical industry to provide multiple properties to consumers. A gas phase to be dispersed in an emulsion must be introduced in such a way as not to destabilize said emulsion. Consequently, for emulsions using a conventional emulsifying surfactant system, either the amount of gas actually dispersed is too low to obtain the advantages of a foam, or the amount of gas actually dispersed is too large and generates instability during storage.

To solve these problems, the prior art discusses ternary emulsifying systems (S_{T}), comprising:
(i) at least one emulsifier (A) selected from saturated or unsaturated, linear or branched, totally or partially salified fatty acids comprising from 10 to 40 carbon atoms,
(ii) at least one emulsifier (B) selected from polyethoxylated fatty acid esters comprising from 10 to 40 carbon atoms and having a degree of ethoxylation of between 5 and 100,
(iii) at least one emulsifier (C) selected from saturated or unsaturated, linear or branched fatty alcohols comprising from 10 to 40 carbon atoms.

The American patent application published under the number US 2004/0161437 A1 describes cosmetic compositions in the form of an emulsion comprising an aqueous phase, at least one lipid phase, an amount of 1% to 90% by volume of gas, an amount by weight of 0.1% to 10% of at least one inorganic gelling agent, an amount by weight of 0.1% to 10% of at least one particulate hydrophobic compound, and the emulsifying system (S_{T}) described above.

The American patent application published under the number US 2004/0197279 A1 describes cosmetic compositions in the form of an emulsion comprising an aqueous phase, at least one lipid phase, an amount of 1% to 90% by volume of gas, an amount by weight of 0.1% to 10% of at least one inorganic gelling agent, and at least one organic hydrocolloid, and the emulsifying system (S_{T}) described above.

The American patent application published under the number US 2009/0017079 A1 describes emulsions in the form of stable foams, the continuous phase of which comprises at least one thickening system and the discontinuous phase of which comprises a structuring agent, such emulsions being prepared by mixing the different phases at a temperature higher than the critical temperature of the thickening system.

The cosmetic emulsions in the form of foams, as described in the prior art, are prepared by carrying out processes involving high technical control, said processes comprising at least one step of bubbling the gas in the emulsion formed beforehand at a determined pressure, generally between 1 and 2.5 bar, at a controlled temperature.

So as to obtain cosmetic emulsions in the form of stable, functional foams suited to the needs of the cosmetic industries, the applicant has therefore developed novel cosmetic emulsions of oil-in-water type in the form of foams and obtained according to a preparation process which does not employ a step of bubbling a gas in said emulsion.

Accordingly, according to a first aspect, a subject of the invention is an emulsion (E₁) of oil-in-water type in the form of a foam, characterized in that it comprises, per 100% of its weight:

a)—from 27% to 89.9% by weight, from 27% to 84.5% by weight, more particularly from 42% to 89.5% by weight and even more particularly from 52.5% to 86.5% by weight of a cosmetically acceptable aqueous phase (A₁);

b)—from 0.1% to 3% by weight, more particularly from 0.5% to 3% by weight and even more particularly from 0.5% to 2.5% by weight of a crosslinked anionic polyelectrolyte (P) resulting from the polymerization, in the presence of at least one crosslinking agent, of partially or totally salified 2-methyl-2-[(1-oxo-2-propenyl)amino]-1-propanesulfonic acid with at least one neutral monomer selected from acrylamide, (2-hydroxyethyl) acrylate, N,N-dialkylacrylamides, in which each of the alkyl groups comprises between one and four carbon atoms, and at least one monomer of formula (I):

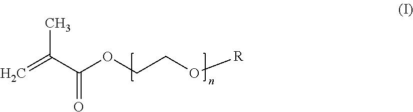

in which R represents a linear or branched alkyl radical comprising from eight to twenty carbon atoms and n represents an integer greater than or equal to one and less than or equal to twenty;

c)—from 5% to 20% by weight, more particularly from 5% to 15% by weight and even more particularly from 8% to 15% by weight of at least one inorganic thickening agent (Ei) selected from natural or synthetic, modified or unmodified phyllosilicates;

d)—from 5% to 50% by weight, more particularly from 5% to 40% by weight and even more particularly from 5% to 30% by weight of a fatty phase (A₂) comprising, per 100% of its weight:
d₁)—from 0.2% to 25% by weight, especially from 0.2% to 20% by weight, more particularly from 0.25% to 12.5% by weight and even more particularly from 0.25% to 10% by weight of an emulsifying system (S) comprising one or more emulsifying surfactants selected from alkylpolyglycoside compositions; alkylpolyglycoside and fatty alcohol compositions; polyethoxylated fatty acids comprising from 10 to 40 carbon atoms and having a degree of ethoxylation of between 5 and 100; compositions of polyethoxylated fatty acids comprising from 10 to 40 carbon atoms and having a degree of ethoxylation of between 5 and 100 and glycerol esters of fatty acids comprising from 10 to 40 carbon atoms; polyethoxylated fatty alcohols comprising from 10 to 40 carbon atoms and having a degree of ethoxylation of between 2 and 100; compositions of polyethoxylated fatty alcohols comprising from 10 to 40 carbon atoms and having a degree of ethoxylation of between 2 and 100 and fatty alcohols comprising from 12 to 40 carbon atoms; compositions of polyethoxylated fatty acids comprising from 10 to 40 carbon atoms and having a degree of ethoxylation of between 5 and 100 and glycerol esters of fatty acids comprising from 10 to 40 carbon atoms and fatty alcohols comprising from 12 to 40 carbon atoms; and
d₂)—from 75% to 99.8% by weight, especially from 80% to 99.8% by weight, more particularly from 87.5% to 99.75% and even more particularly from 90% to 99.75% by weight of at least one oil and optionally at least one wax;
   said emulsion (E₁) being further characterized in that the weight ratio between the inorganic thickening agent (Ei) and the crosslinked anionic polyelectrolyte (P) is greater than or equal to 6/1 and less than or equal to 20/1, more particularly greater than or equal to 8/1 and less than or equal to 15/1 and even more particularly greater than or equal to 10/1 and less than or equal to 15/1.

The expression “cosmetically acceptable” used in the definition of the aqueous phase (A₁) present in the emulsion (E₁) which is the subject of the present invention means, according to the Council of the European Economic Community Directive no. 76/768/EEC of Jul. 27, 1976, amended by Directive no. 93/35/EEC of Jun. 14, 1993, that it includes any substance or preparation intended to be brought into contact with the various parts of the human body (epidermis, body hair and head hair system, nails, lips and genitals) or with the teeth and mucous membranes of the mouth, for the purpose, exclusively and mainly, of cleansing them, fragrancing them, modifying the appearance thereof and/or correcting body odors thereof and/or protecting them or keeping them in good condition.

According to a particular aspect, the emulsion (E₁) as defined above comprises, per 100% of its weight:

a)—from 52.5% to 80% by weight of said aqueous phase (A₁).

In the definition of the emulsion (E₁) which is a subject of the present invention, “oil” denotes a compound and/or a mixture of compounds insoluble in water and liquid at 25° C., and more particularly:

mineral oils such as paraffin oil, liquid petroleum jelly, isoparaffins or white mineral oils;

oils of animal origin, such as squalene or squalane;

vegetable oils, such as phytosqualane, sweet almond oil, coconut oil, castor oil, jojoba oil, olive oil, rapeseed oil, peanut oil, sunflower oil, wheat germ oil, corn germ oil, soybean oil, cottonseed oil, alfalfa oil, poppy seed oil, pumpkin seed oil, evening primrose oil, millet oil, barley oil, rye oil, safflower oil, candlenut oil, passionflower oil, hazelnut oil, palm oil, shea butter, apricot kernel oil, calophyllum oil, sisymbrium oil, avocado oil, calendula oil, oils derived from flowers or vegetables;

ethoxylated vegetable oils;

synthetic oils such as fatty acid esters such as butyl myristate, propyl myristate, isopropyl myristate, cetyl myristate, isopropyl palmitate, octyl palmitate, butyl stearate, hexadecyl stearate, isopropyl stearate, octyl stearate, isocetyl stearate, dodecyl oleate, hexyl laurate, propylene glycol dicaprylate, esters derived from lanolic acid, such as isopropyl lanolate, isocetyl lanolate, fatty acid monoglycerides, diglycerides and triglycerides such as glycerol triheptanoate, alkylbenzoates, hydrogenated oils, poly(alpha-olefin)s, polyolefins such as poly(isobutane), synthetic isoalkanes such as isohexadecane, isododecane, perfluorinated oils and

silicone oils such as dimethylpolysiloxanes, methylphenylpolysiloxanes, amine-modified silicones, fatty acid-modified silicones, alcohol-modified silicones, silicones modified with alcohols and fatty acids, silicones modified with polyether groups, epoxy-modified silicones, silicones modified with fluoro groups, cyclic silicones and silicones modified with alkyl groups.

According to another particular aspect, the emulsion (E₁) as defined above comprises, per 100% of its weight:

d)—from 15% to 40% by weight and more particularly from 15% to 30% by weight of said fatty phase (A₂).

When the emulsion (E₁) as defined above comprises a wax, the latter is more particularly selected from beeswax, carnauba wax, candelilla wax, ouricoury wax, Japan wax, cork fiber wax, sugar cane wax, paraffin waxes, lignite waxes, microcrystalline waxes, lanolin wax; ozokerite; polyethylene wax; silicone waxes; vegetable waxes; fatty alcohols and fatty acids that are solid at room temperature; glycerides that are solid at room temperature.

In the definition of the emulsion (E₁) which is a subject of the present invention, crosslinked anionic polyelectrolyte (P) denotes a non-linear crosslinked anionic polyelectrolyte, which is in the form of a three-dimensional network which is insoluble in water, but swellable in water and which leads to the production of a chemical gel.

According to another particular aspect, the emulsion (E₁) as defined above comprises, per 100% of its weight:

a)—from 27% to 84.5% by weight of said aqueous phase (A₁);

b)—from 0.5% to 3% by weight of said crosslinked anionic polyelectrolyte (P).

In the definition of the crosslinked anionic polyelectrolyte (P) present in the emulsion (E₁) as defined above, partially or totally salified means that said 2-methyl-2-[(1-oxo-2-propenyl)amino]-1-propanesulfonic acid is partially or totally salified, especially in the form of an alkali metal salt, for example in the form of a sodium salt or a potassium salt, or in the form of an ammonium salt.

In the definition of the emulsion (E₁) which is a subject of the present invention, neutral monomer selected from N,N-dialkylacrylamides, in which each of the alkyl groups comprises between one and four carbon atoms, denotes more particularly N,N-dimethylacrylamide, N,N-diethylacrylamide, N,N-dipropylacrylamide, or N,N-diisopropylacrylamide.

According to a particular aspect of the present invention, in the emulsion (E₁) as defined above, said crosslinked anionic polyelectrolyte (P) comprises, per 100 mol % of its constituent monomers, from 5 mol % to 95 mol %, more particularly from 10 mol % to 90 mol %, and most particularly from 20 mol % to 80 mol % of monomer units derived from partially or totally salified 2-methyl-2-[(1-oxo-2-propenyl)amino]-1-propanesulfonic acid.

According to another particular aspect of the present invention, in the emulsion (E₁) as defined above, said crosslinked anionic polyelectrolyte (P) comprises, per 100 mol % of its constituent monomers, from 4.9 mol % to 90 mol %, more particularly from 9.5 mol % to 85 mol %, and most particularly from 15 mol % to 75 mol % of monomer units derived from at least one neutral monomer selected from acrylamide, (2-hydroxyethyl) acrylate, N,N-dialkylacrylamides, in which each of the alkyl groups comprises between one and four carbon atoms, in which each of the alkyl groups comprises between one and four carbon atoms.

According to another particular aspect of the present invention, in the emulsion (E₁) which is a subject of the present invention, said crosslinked anionic polyelectrolyte (P) as defined above comprises, per 100 mol % of its constituent monomers, from 0.1 mol % to 10 mol % and more particularly from 0.5 mol % to 5 mol % of monomer units derived from the monomer of formula (I).

A subject of the invention is more particularly an emulsion (E₁) as defined above, characterized in that said crosslinked anionic polyelectrolyte (P) comprises, per 100 mol % of its constituent monomers:

from 20 mol % to 80 mol % of monomer units derived from said partially or totally salified 2-methyl-2-[(1-oxo-2-propenyl)amino]-1-propanesulfonic acid;

from 15 mol % to 75 mol % of monomer units derived from at least one neutral monomer chosen from acrylamide, (2-hydroxyethyl) acrylate, N,N-dialkylacrylamides, in which each of the alkyl groups comprises between one and four carbon atoms,

from 0.5 mol % to 5 mol % of monomer units derived from said monomer of formula (I) as defined above.

In the formula (I) of the monomer present in said crosslinked anionic polyelectrolyte (P) included in the emulsion (E₁) which is a subject of the present invention, linear or branched alkyl radical comprising from eight to twenty carbon atoms more particularly denotes, for R:

either a radical derived from linear primary alcohols such as, for example, the octyl, decyl, undecyl, dodecyl, tridecyl, tetradecyl, pentadecyl, hexadecyl, heptadecyl, octadecyl, nonadecyl or eicosyl radical;

or a radical derived from Guerbet alcohols, which are branched 1-alkanols corresponding to the general formula:

CH₃—(CH₂)ₚ—CH[CH₃—(CH₂)ₚ₋₂]—CH₂OH,

in which p represents an integer between 2 and 9, such as, for example, 2-ethylhexyl, 2-propylheptyl, 2-butyloctyl, 2-pentylnonyl, 2-hexyldecyl or 2-octyldodecyl radicals;

or a radical derived from isoalkanols corresponding to the general formula:

CH₃—CH(CH₃)—(CH₂)ₘ—CH₂OH,

in which m represents an integer between 2 and 16, such as, for example, 4-methylpentyl, 5-methylhexyl, 6-methylheptyl, 15-methylpentadecyl or 16-methylheptadecyl radicals, or 2-hexyloctyl, 2-octyldecyl or 2-hexyldodecyl radicals.

According to another particular aspect, the emulsion (E₁) as defined above is characterized in that said neutral monomer is selected from acrylamide, (2-hydroxyethyl) acrylate or N,N-dimethylacrylamide.

According to another even more particular aspect, the emulsion (E₁) as defined above is characterized in that said neutral monomer is N,N-dimethylacrylamide.

According to another particular aspect, in the formula (I) as defined above, R represents an alkyl radical comprising from 12 to 18 carbon atoms.

According to an even more particular aspect, a subject of the invention is an emulsion (E₁) as defined above, characterized in that in formula (I) as defined above, R represents an alkyl radical selected from the dodecyl, tridecyl, tetradecyl, hexadecyl or octadecyl radical.

According to another particular aspect, a subject of the invention is a composition (E₁) as defined above, characterized in that in formula (I) as defined above, n represents an integer greater than or equal to 3 and less than or equal to 20.

According to an even more particular aspect, the emulsion (E₁) as defined above is characterized in that said monomer of formula (I) is tetraethoxylated lauryl methacrylate.

According to another particular aspect, a subject of the invention is an emulsion (E₁) as defined above, characterized in that said crosslinked anionic polyelectrolyte (P) as defined above is crosslinked with a diethylenic or polyethylenic compound in the molar proportion, expressed relative to the monomers used, is from 0.005 mol % to 1 mol %, more particularly from 0.01 mol % to 0.5 mol % and most particularly from 0.01 mol % to 0.25 mol%. The crosslinking agent is more particularly selected from ethylene glycol dimethacrylate, tetraallyloxyethane, ethylene glycol diacrylate, diallyl urea, triallylamine, trimethylolpropane triacrylate or methylenebis(acrylamide) or a mixture of these compounds.

In the process for preparing the crosslinked anionic polyelectrolyte (P) used in the emulsion (E₁) which is a subject of the present invention, various additives such as complexing agents, transfer agents or chain-limiting agents may also be employed during the polymerization reaction. The transfer agents or chain-limiting agents are more particularly selected from the group consisting of sodium hypophosphite, alcohols of low molecular weight, for example methanol, ethanol, 1-propanol, isopropanol, butanol, thiols, for example 2-mercaptoethanol, transfer agents comprising a sulfate function, for example sodium methallyl sulfonate, or mixtures of said transfer agents. The transfer agents or chain-limiting agents are more particularly used in molar proportions, expressed relative to the total number of moles of monomers used, of 0.001 mol % to 1 mol %, more particularly of 0.001 mol % to 0.5 mol %, and most particularly from 0.001 mol % to 0.1 mol%.

According to one particular aspect, a subject of the invention is an emulsion (E₁) as defined above, characterized in that said crosslinked anionic polyelectrolyte (P) as defined above is selected from the terpolymers of 2-methyl-2-[(1-oxo-2-propenyl)amino]-1-propanesulfonic acid, partially salified in ammonium salt form, of N,N-dimethylacrylamide and of tetraethoxylated lauryl methacrylate, crosslinked with trimethylolpropane triacrylate, or from the terpolymers of 2-methyl-2-[(1-oxo-2-propenyl)amino]-1-propanesulfonic acid, partially salified in ammonium salt form, of N,N-dimethylacrylamide and of eicosaethoxylated stearyl methacrylate, crosslinked with trimethylolpropane triacrylate.

According to a more particular aspect, a subject of the invention is an emulsion (E₁) as defined above, characterized in that said crosslinked anionic polyelectrolyte (P) is a terpolymer of 2-methyl-2-[(1-oxo-2-propenyl)amino]-1-propanesulfonic acid, partially salified in ammonium salt form, of N,N-dimethylacrylamide and of tetraethoxylated lauryl methacrylate, crosslinked with trimethylolpropane triacrylate.

According to another more particular aspect, a subject of the invention is an emulsion (E₁) as defined above, characterized in that said crosslinked anionic polyelectrolyte (P) comprises, per 100 mol%:

from 60 mol % to 80 mol % of monomer units derived from 2-methyl-2-[(1-oxo-2-propenyl)amino]-1-propanesulfonic acid, partially salified in ammonium form,

from 15 mol % to 39.5 mol % of monomer units derived from N,N-dimethylacrylamide, and

from 0.5 mol % to 5 mol % of monomer units derived from tetraethoxylated lauryl methacrylate.

According to another particular aspect, the emulsion (E₁) as defined above comprises, per 100% of its weight:

b)—from 0.5% to 2.5% by weight of said crosslinked anionic polyelectrolyte (P).

In the definition of the inorganic thickening agent (E₁) present in the emulsion (E₁) which is a subject of the present invention, the term “phyllosilicate” denotes any compound which is a mineral of the group of the silicates constructed by stacking tetrahedral layers (“T”), where the tetrahedra share three vertices out of four (“basal” oxygens), the fourth vertex (“apical” oxygen) being connected to an octahedral layer (“O”) occupied by different cations, for example aluminum, magnesium, iron, titanium or lithium cations. The union of the T layers and the O layers forms sheets which represent the cleavage unit of the phyllosilicates.

According to another particular aspect, a subject of the invention is an emulsion (E₁) as defined above, characterized in that the inorganic thickening agent (Ei) is a natural or synthetic, modified or unmodified phyllosilicate selected from aluminum silicates, magnesium silicates, or aluminum magnesium silicates.

According to a more particular aspect, a subject of the invention is an emulsion (E₁) as defined above, characterized in that the inorganic thickening agent (Ei) is selected from kaolinite, montmorillonite, illite, beidellite, saponite, bentonite, hectorite, vermiculite, serpentine, nacrite, amesite, nontronite, lizardite, sericite, halloysite, muscovite, paragonite, damouzite, glauconite or celadonite.

According to an even more particular aspect, a subject of the invention is an emulsion (E₁) as defined above, characterized in that the inorganic thickener (Ei) is selected from kaolinite, montmorillonite, saponite, bentonite or hectorite.

According to another particular aspect, the emulsion (E₁) as defined above comprises, per 100% of its weight:

c)—from 10% to 15% by weight of said inorganic thickening agent (Ei).

According to another particular aspect, in the emulsion (E₁) as defined above, the weight ratio between said inorganic thickening agent (Ei) and said crosslinked anionic polyelectrolyte (P) is greater than or equal to 8/1.

In the definition of the emulsion (E₁) which is a subject of the present invention, “alkylpolyglycoside composition included in the emulsifying system (S)” is used to denote a composition (C₁) represented by the formula (II):

R₁—O-(G)ₓ-H (II)

in which x represents a decimal number between 1.05 and 5, G represents the residue of a reducing sugar, and R₁ represents a saturated or unsaturated, linear or branched aliphatic hydrocarbon-based radical optionally substituted with one or more hydroxyl groups, comprising from 12 to 36 carbon atoms, said composition (C₁) consisting of a mixture of compounds represented by the formulae (I₁), (I₂), (I₃), (I₄) and (I₅):

R₁—O-(G)₁-H (II₁)

R₁—O-(G)₂-H (II₂)

R₁—O-(G)₃-H (II₃)

R₁—O-(G)₄-H (II₄)

R₁—O-(G)₅-H (II₅)

in the respective molar proportions a₁, a₂, a₃, a₄ and a₅, such that:

the sum a₁+a₂+a₃+a₄+a₅ is equal to 1, and that

the sum a₁+2a₂+3a₃+4a₄+5a₅ is equal to x.

“Saturated or unsaturated, linear or branched aliphatic hydrocarbon-based radical comprising from 12 to 36 carbon atoms, optionally substituted with one or more hydroxyl groups” is used to denote, for the radical R₁ in formula (II) as defined above:

saturated linear alkyl radicals, for example n-dodecyl, n-tridecyl, n-tetradecyl, n-pentadecyl, n-hexadecyl, n-heptadecyl, n-octadecyl, n-nonadecyl, n-eicosyl, n-docosyl radicals;

unsaturated linear radicals such as dodecenyl, tridecenyl, tetradecenyl, pentadecenyl, hexadecenyl, heptadecenyl, octadecenyl, nonadecenyl, eicosenyl, docosenyl, 4-dodecenyl or 5-dodecenyl radicals;

saturated or unsaturated, linear or branched aliphatic radicals comprising from 12 to 36 carbon atoms, substituted with one or two hydroxyl groups, such as hydroxydodecyl, hydroxytetradecyl, hydroxyhexadecyl, hydroxyoctadecyl, hydroxyeicosyl or hydroxydocosyl radicals, for example the 12-hydroxyoctadecyl radical;

radicals derived from isoalkanols of formula (1):

(CH₃)(CH₃)CH—(CH₂)ᵣ—CH₂—OH (1)

in which r represents an integer between 8 and 20, for example isodecyl, isoundecyl, isododecyl, isotridecyl, isotetradecyl, isopentadecyl, isohexadecyl, isopentadecyl, isooctadecyl, isononadecyl, isoeicosyl or isodocosyl radicals; branched alkyl radicals, derived from Guerbet alcohols, of formula (2):

CH(CₛH₂ₛ₊₁)(CₜH₂ₜ₊₁)—CH₂—OH (2)

in which t is an integer between 6 and 18, s is an integer between 4 and 18 and the sum s+t is greater than or equal to 10 and less than or equal to 22, for example 2-butyloctyl, 2-butyldecyl, 2-hexyloctyl, 2-hexyldecyl, 2-octyldecyl, 2-hexyldodecyl, 2-octyldodecyl, 2-decyltetradecyl, 2-dodecylhexadecyl or 2-tetradecyloctadecyl radicals. According to a particular aspect, in the definition of formula (II) as defined above, R₁ represents a saturated or unsaturated, linear or branched aliphatic hydrocarbon-based radical comprising from 12 to 24 carbon atoms.

The term “reducing sugar” in the definition of formula (II) as defined above denotes saccharide derivatives that do not have in their structures any glycoside bond established between an anomeric carbon and the oxygen of an acetal group as defined in the reference publication: “Biochemistry”, Daniel Voet/Judith G. Voet, p. 250, John Wyley & Sons, 1990. The oligomeric structure (G)ₓ may be in any isomeric form, whether it is optical isomerism, geometrical isomerism or positional isomerism; it may also represent a mixture of isomers.

In formula (II) as defined above, the group R₁—O— is linked to G via the anomeric carbon of the saccharide residue, so as to form an acetal function.

According to a particular aspect in the definition of formula (II) as defined above, G represents the residue of a reducing sugar selected from glucose, dextrose, sucrose, fructose, idose, gulose, galactose, maltose, isomaltose, maltotriose, lactose, cellobiose, mannose, ribose, xylose, arabinose, lyxose, allose, altrose, dextran and tallose; and more particularly G represents the residue of a reducing sugar selected from glucose, xylose and arabinose residues.

According to an even more particular aspect, in the definition of formula (II) representing composition (C₁) included in the emulsion E₁ that is a subject of the present invention, x represents a decimal number greater than or equal to 1.05 and less than or equal to 2.5, more particularly greater than or equal to 1.05 and less than or equal to 2.0 and even more particularly greater than or equal to 1.25 and less than or equal to 2.0

According to an even more particular aspect, the emulsion (E₁) as defined above is characterized in that, in the definition of said emulsifying system (S), the alkylpolyglycoside compositions are compositions (C1) represented by formula (II):

R1-O-(G)ₓ-H (II)

in which x represents a decimal number between 1.05 and 2.5, G represents the residue of a reducing sugar, and R1 represents a linear or branched, saturated or unsaturated aliphatic hydrocarbon-based radical comprising from 12 to 36 carbon atoms, said composition (C1) consisting of a mixture of compounds represented by the formulae (I₁), (I₂), (I₃), (I₄) and (I₅):

R1-O-(G)₁-H (II₁)

R1-O-(G)₂-H (II₂)

R1-O-(G)₃-H (II₃)

R1-O-(G)₄-H (II₄)

R1-O-(G)₅-H (II₅)

in the respective molar proportions a₁, a₂, a₃, a₄ and a₅, such that:

the sum a₁+a₂+a₃+a₄+a₅ is equal to 1, and that

the sum a₁+2a₂+3a₃+4a₄+5a₅ is equal to x.

According to an even more particular aspect, in the definition of formula (II) as defined above, R₁ represents the radical selected from at least one of the elements of the group consisting of n-dodecyl, n-tetradecyl, n-hexadecyl, n-octadecyl, n-eicosyl, n-docosyl, n-oleyl, n-linoleyl, n-linolenyl, 2-hexyldecyl, 2-octyldecyl, 2-hexyldodecyl, 2-octyldodecyl and 2-decyltetradecyl radicals; G represents the residue of a reducing sugar selected from glucose and xylose residues and x represents a decimal number greater than or equal to 1.05 and less than or equal to 2.5.

According to an even more particular aspect, the emulsion as defined above is characterized in that in the composition (C1) represented in the formula (II), x represents a decimal number between 1.05 and 2.5, G represents the glucose residue and R1 represents an aliphatic hydrocarbon-based radical selected from n-dodecyl, n-tetradecyl, n-hexadecyl, n-octadecyl, n-eicosyl, n-docosyl, n-oleyl, n-linoleyl or n-linolenyl radicals.

According to an even more particular aspect, a subject of the invention is an emulsion (E₁) as defined above, characterized in that, in the definition of said emulsifying system (S), the alkylpolyglycoside and fatty alcohol compositions are compositions (C2) comprising, per 100% of their weight:

from 10% to 60% by weight of at least one composition (C1) represented by formula (II):

R1-O-(G)ₓ-H (II)

in which x represents a decimal number between 1.05 and 2.5, G represents the residue of a reducing sugar, and R1 represents a linear or branched, saturated or unsaturated aliphatic hydrocarbon-based radical comprising from 12 to 36 carbon atoms, said composition (C1) consisting of a mixture of compounds represented by the formulae (I₁), (I₂), (I₃), (I₄) and (I₅):

R1-O-(G)₁-H (II₁)

R1-O-(G)₂-H (II₂)

R1-O-(G)₃-H (II₃)

R1-O-(G)₄-H (II₄)

R1-O-(G)₅-H (II₅)

in the respective molar proportions a₁, a₂, a₃, a₄ and a₅, such that:

the sum a₁+a₂+a₃+a₄+a₅ is equal to 1, and

the sum a₁+2a₂+3a₃+4a₄+5a₅ is equal to x;

from 40% to 90% by weight of at least one fatty alcohol of formula (III):

R′1—OH (III),

in which R′1 represents a linear or branched, saturated or unsaturated aliphatic hydrocarbon-based radical comprising from 12 to 40 carbon atoms.

According to an even more particular aspect, a subject of the invention is an emulsion (E₁) as defined above, characterized in that said emulsifying system (S) consists of a composition (C₂) for which:

in the composition (C₁) represented by the formula (II), x represents a decimal number between 1.05 and 2.5, G represents the residue of a reducing sugar selected from glucose, xylose and arabinose, and R1 represents an aliphatic hydrocarbon-based radical selected from n-dodecyl, n-tetradecyl, n-hexadecyl, n-octadecyl, n-eicosyl, n-docosyl, n-oleyl, n-linoleyl or n-linolenyl radicals;

in the formula (III), R′1 represents an aliphatic hydrocarbon-based radical selected from n-dodecyl, n-tetradecyl, n-hexadecyl, n-octadecyl, n-eicosyl, n-docosyl, n-oleyl, n-linoleyl or n-linolenyl radicals.

According to an even more particular aspect, a subject of the invention is an emulsion (E₁) as defined above, characterized in that:

in the composition of formula (C1) represented by the formula (II), x represents a decimal number between 1.05 and 2.5, G represents the glucose residue, and R 1 represents a radical selected from dodecyl, tetradecyl, hexadecyl, octadecyl, eicosyl, behenyl, oleyl, linoleyl or linolenyl radicals; and

in the formula (III), R′1 represents an aliphatic hydrocarbon-based radical selected from dodecyl, tetradecyl, hexadecyl, octadecyl, eicosyl, behenyl, oleyl, linoleyl or linolenyl radicals.

In the definition of the emulsion (E₁) which is a subject of the present invention, “polyethoxylated fatty acids comprising from 10 to 40 carbon atoms and having a degree of ethoxylation of between 5 and 100” denotes a compound of formula (IV):

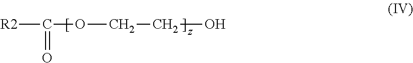

in which:

R2-C(═O) represents a saturated or unsaturated, linear or branched acyl radical containing from 10 to 40 carbon atoms, and more particularly a radical selected from n-decanoyl, n-dodecanoyl, n-tetradecanoyl, n-hexadecanoyl, n-octadecanoyl, n-eicosanoyl, n-docosanoyl, n-oleyl, n-linoleyl, n-linolenoyl or isostearyl radicals, and

z represents an integer greater than or equal to 5 and less than or equal to 100, and more particularly greater than or equal to 5 and less than or equal to 80.

According to a more particular aspect, the compound of formula (IV) is selected from PEG-5 stearate, PEG-9 stearate, PEG-20 stearate, PEG-30 stearate, PEG-40 stearate, PEG-50 stearate, PEG-60 stearate, PEG-80 stearate, PEG-100 stearate, PEG-5 palmitate, PEG-9 palmitate, PEG-20 palmitate, PEG-30 palmitate, PEG-40 palmitate, PEG-50 palmitate, PEG-60 palmitate, PEG-80 palmitate, PEG-100 palmitate, PEG-5 laurate, PEG-9 laurate, PEG-20 laurate, PEG-30 laurate, PEG-40 laurate, PEG-50 laurate, PEG-60 laurate, PEG-80 laurate, PEG-100 laurate, PEG-5 oleate, PEG-9 oleate, PEG-20 oleate, PEG-30 oleate, PEG-40 oleate, PEG-50 oleate, PEG-60 oleate, PEG-80 oleate and PEG-100 oleate.

In the definition of the emulsion (E₁) which is a subject of the present invention, “glycerol ester of fatty acids comprising from 10 to 40 carbon atoms” denotes a compound of formula (V):

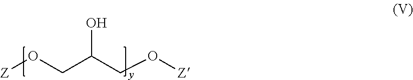

in which Z represents an acyl radical of formula R₃—C(═O)—, in which R₃ represents a saturated or unsaturated, linear or branched aliphatic hydrocarbon-based radical comprising from 9 to 39 carbon atoms and more particularly an acyl radical selected from n-lauroyl, n-dodecanoyl, n-tetradecanoyl, n-hexadecanoyl, n-octadecanoyl, n-eicosanoyl, n-docosanoyl, n-oleyl, n-linoleyl, n-linolenyl, or isostearyl radicals, Z′ represents the acyl radical of formula R₃—C(═O)— as defined above, with Z′ being identical to or different from Z, or a hydrogen atom, and y represents an integer greater than or equal to 2 and less than or equal to 20.

According to a more particular aspect, the compound of formula (V) is selected from decaglycerol oleate, decaglycerol isostearate, decaglycerol monolaurate, decaglycerol mono-linoleate and decaglycerol mono-myristate.

In the definition of the emulsion (E₁) which is a subject of the present invention, “polyethoxylated fatty alcohol comprising from 10 to 40 carbon atoms and having a degree of ethoxylation of between 2 and 100” denotes a compound of formula (VI):

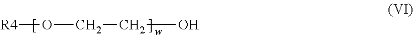

in which:

R4 represents a saturated or unsaturated, linear or branched aliphatic hydrocarbon-based radical comprising from 10 to 40 carbon atoms, optionally substituted with one or more hydroxyl groups. For the radical R₄ in the formula (VI) as defined above, the following are denoted: saturated linear alkyl radicals, for example n-decyl, n-dodecyl, n-tridecyl, n-tetradecyl, n-pentadecyl, n-hexadecyl, n-heptadecyl, n-octadecyl, n-nonadecyl, n-eicosyl, n-docosyl radicals; unsaturated linear radicals such as dodecenyl, tridecenyl, tetradecenyl, pentadecenyl, hexadecenyl, heptadecenyl, octadecenyl, nonadecenyl, eicosenyl, docosenyl, 4-dodecenyl or 5-dodecenyl radicals; saturated or unsaturated, linear or branched aliphatic radicals comprising from 10 to 40 carbon atoms substituted with one or two hydroxyl groups, such as hydroxydodecyl, hydroxytetradecyl, hydroxyhexadecyl, hydroxyoctadecyl, hydroxyeicosyl or hydroxydocosyl radicals, for example the 12-hydroxyoctadecyl radical; radicals derived from isoalkanols, for example isodecyl, isoundecyl, isododecyl, isotridecyl, isotetradecyl, isopentadecyl, isohexadecyl, isopentadecyl, isooctadecyl, isononadecyl, isoeicosyl or isodocosyl radicals; branched alkyl radicals derived from Guerbet alcohols, for example 2-butyloctyl, 2-butyldecyl, 2-hexyloctyl, 2-hexyldecyl, 2-octyldecyl, 2-hexyldodecyl, 2-octyldodecyl, 2-decyltetradecyl, 2-dodecylhexadecyl or 2-tetradecyloctadecyl radicals,

w represents an integer greater than or equal to 2 and less than or equal to 100, more particularly greater than or equal to 2 and less than or equal to 80 and even more particularly greater than or equal to 2 and less than or equal to 50.

In the definition of the emulsion (E₁) which is a subject of the present invention, “composition of polyethyoxylated fatty alcohol comprising from 10 to 40 carbon atoms and having a degree of ethoxylation of between 2 and 100 and of fatty alcohol comprising from 12 to 40 carbon atoms” denotes the composition (C₃) comprising, per 100% of its weight:

from 20% to 80%, more particularly from 30% to 70%, and even more particularly from 40% to 60% by weight of at least one compound of formula (VI) as defined above

from 80% to 20%, more particularly from 70% to 30% by weight, and even more particularly from 60% to 40% by weight of at least one fatty alcohol of formula (III) as defined above.

In the definition of the emulsion (E₁) which is a subject of the present invention, “composition of polyethyoxylated fatty acid comprising from 10 to 40 carbon atoms and having a degree of ethoxylation of between 5 and 100 and of glycerol ester of fatty acid comprising from 10 to 40 carbon atoms and of fatty alcohol comprising from 12 to 40 carbon atoms” denotes the composition (C₄) comprising, per 100% of its weight:

from 10% to 40%, more particularly from 15% to 35%, and even more particularly from 20% to 30% by weight of at least one compound of formula (IV) as defined above, and from 10% to 40%, more particularly from 15% to 35% and even more particularly from 20% to 30% by weight of at least one compound of formula (V) as defined above, and

from 20% to 80%, more particularly from 30% to 70%, and even more particularly from 40% to 60% by weight of at least one compound of formula (III) as defined above.

According to another particular aspect, a subject of the invention is the emulsion (E₁) as defined above, characterized in that it also comprises, per 100% of its weight,

(e)—a proportion by weight greater than or equal to 0.01% and less than or equal to 0.5% of at least one polysaccharide (PS) selected from polysaccharides consisting of monosaccharide derivatives and polysaccharides consisting solely of monosaccharides.

Polysaccharides (PS) are saccharide polymers. The IUPAC definition of the saccharides designates monosaccharides, compounds of monosaccharides proper and their derivatives, obtained either by reduction of a carbonyl group, or by oxidation of one or more hydroxyl functions, or by the replacement of one or more hydroxyl functions with a hydrogen atom, an amine group, a phosphate function, or a sulfate function.

The polysaccharides most commonly used for preparing food, cosmetic or pharmaceutical compositions predominantly consist of monosaccharides, such as glucose, galactose, mannose or of monosaccharide derivatives for which the hydroxyl function of the terminal carbon has been oxidized to give a carboxyl function. Consequently, two distinct groups may be distinguished among the polysaccharides: polysaccharides consisting solely of monosaccharides (or poly-monosaccharides) and polysaccharides consisting of monosaccharide derivatives.

Among the polysaccharides composed solely of monosaccharides, a distinction may be made between glucans, which are homopolymers of glucose which are very abundant in nature, glucomannoglycans, xyloglycans and galactomannans, which are polymers, the main chain of which consists of D-mannose units, joined together at β-1,4, and on which D-galactose units are grafted laterally by α-1,6 bonds.

Galactomannans are present in several plant species, and more particularly in the leguminous species in which they constitute the albumen of the seeds. Depending on their plant origin, the degree of substitution (DS) of the D-galactose units on the D-mannose main chain of galactomannans varies between 0 and 1:

galactomannans originating from cassia gum have a degree of substitution (DS) of approximately 1/5, meaning the lateral grafting of one D-galactose unit every 5 D-mannose units present on the main chain of the polysaccharide;

galactomannans originating from locust bean gum have a degree of substitution (DS) of approximately 1/4, meaning the lateral grafting of one D-galactose unit every 4 D-mannose units present on the main chain of the polysaccharide;

galactomannans originating from tara gum have a degree of substitution (DS) of approximately 1/3, meaning the lateral grafting of one D-galactose unit every 3 D-mannose units present on the main chain of the polysaccharide;

galactomannans originating from guar gum have a degree of substitution (DS) of approximately 1/2, meaning the lateral grafting of one D-galactose unit every 2 D-mannose units present on the main chain of the polysaccharide;

galactomannans originating from fenugreek gum have a degree of substitution (DS) of approximately 1/1, meaning the lateral grafting of one D-galactose unit for practically every D-mannose unit for practically present on the main chain of the polysaccharide.

Among the polysaccharides consisting of monosaccharide derivatives, a distinction may be made between:

sulfated galactans, which are polymers of galactose which may have pendent sulfate-ester groups, represented especially by algal polysaccharides such as carrageenans and agar;

uronans, which are the polymers of uronic acids such as algins and pectins;

heteropolymers of monosaccharides and uronic acids: often of complex composition, these polymers are found especially in sap exudates (such as, for example, the exudate of gum arabic and the exudate of gum karaya), but they are also produced by microorganisms, such as, for example, xanthan gum and gellan gum;

glucosaminoglycans which are polysaccharides formed from a glucose derived by replacing its C-2 hydroxyl with an amine (referred to as 2-amino-2-deoxy-D-glucose or, more simply, glucosamine). The amine function can also be acetylated.

Among the hydrocolloids in this class are chitosan, formed solely of glucosamine units, and hyaluronan, the repeat unit of which is a dimer of glucosamine and glucuronic acid.

According to a more particular aspect, a subject of the invention is the emulsion (E₁) as defined above, characterized in that the polysaccharide (PS) is selected from glucans, glucomannoglycans, xyloglycans and galactomannans, sulfated galactans, uronans, heteropolymers of monosaccharides and uronic acids, and glucosaminoglycans.

According to a more particular aspect, a subject of the invention is the emulsion (E₁) as defined above, characterized in that the polysaccharide (PS) is selected from the group consisting of carrageenans, agar, exudate of gum arabic, exudate of gum karaya, xanthan gum, gellan gum, chitosan, glucoans, galactomannans originating from cassia gum, galactomannans originating from locust bean gum, galactomannans originating from tara gum, galactomannans originating from guar gum and galactomannans originating from fenugreek gum.

As a particular example of such an emulsion (E₁) as defined above, there is that which is characterized in that it comprises, per 100% of its weight:

a)—from 52.5% to 76.4% by weight of a cosmetically acceptable aqueous phase (A₁);

b)—from 0.5% to 2% by weight of a crosslinked anionic polyelectrolyte (P) resulting from the polymerization, in the presence of at least one crosslinking agent, of from 60 mol % to 80 mol % of monomer units derived from 2-methyl-2-[(1-oxo-2-propenyl)amino]-1-propanesulfonic acid, partially or totally salified in ammonium salt form, with from 15 mol % to 39.5 mol % of monomer units derived from N,N-dimethylacrylamide, and from 0.5 mol % to 5 mol % of monomer units derived from tetraethoxylated lauryl methacrylate,

c)—from 8% to 15% by weight of at least one inorganic thickening agent (Ei) selected from among kaolinite, montmorillonite, saponite, bentonite or hectorite,

d)—from 15% to 30% by weight of a fatty phase (A₂) comprising, per 100% of its weight:
d₁)—from 5% to 25% by weight of an emulsifying system (S) comprising one or more emulsifying surfactants selected from alkylpolyglycoside compositions; alkylpolyglycoside and fatty alcohol compositions; polyethoxylated fatty acids comprising from 10 to 40 carbon atoms and having a degree of ethoxylation of between 5 and 100; compositions of polyethoxylated fatty acids comprising from 10 to 40 carbon atoms and having a degree of ethoxylation of between 5 and 100 and glycerol esters of fatty acids comprising from 10 to 40 carbon atoms; polyethoxylated fatty alcohols comprising from 10 to 40 carbon atoms and having a degree of ethoxylation of between 2 and 100; compositions of polyethoxylated fatty alcohols comprising from 10 to 40 carbon atoms and having a degree of ethoxylation of between 2 and 100 and fatty alcohols comprising from 12 to 40 carbon atoms; compositions of polyethoxylated fatty acids comprising from 10 to 40 carbon atoms and having a degree of ethoxylation of between 5 and 100 and glycerol esters of fatty acids comprising from 10 to 40 carbon atoms and fatty alcohols comprising from 12 to 40 carbon atoms; and
d₂)—from 75% to 95% by weight of at least one oil and optionally at least one wax;

e)—from 0.1% to 0.5% by weight of at least one polysaccharide selected from agar, exudate of gum arabic, exudate of gum karaya, xanthan gum, gellan gum, galactomannans originating from cassia gum, galactomannans originating from locust bean gum, galactomannans originating from tara gum, galactomannans originating from guar gum and galactomannans originating from fenugreek gum, said emulsion (E₁) being further characterized in that the weight ratio between the inorganic thickening agent (Ei) and the crosslinked anionic polyelectrolyte (P) is greater than or equal to 8/1 and less than or equal to 20/1.

The emulsion (E₁) which is a subject of the present invention as defined above is intended for topical use and may be incorporated in any type of cosmetic, dermocosmetic, dermopharmaceutical or pharmaceutical formulation intended for topical use or else in any type of support intended to be placed in contact with the skin (paper, wipe, textile, transdermal device, etc.).

The expression “for topical use” means that the emulsion (E₁) is used by application to the skin, hair, scalp or mucous membranes, whether this is a direct application in the case of a cosmetic, dermocosmetic, dermopharmaceutical or pharmaceutical formulation or an indirect application, for example in the case of a product for bodily hygiene or for caring for or protecting the skin, in the form of a textile article, for example a wipe, or a paper article, for example a paper for sanitary use.

The emulsion (E₁) which is a subject of the present invention may be packaged in a bottle with a total or partial opening, in a device of “pump-bottle” type, in a device equipped with a perforated wall, for example a grille, or in a device equipped with a ball applicator (known as a “roll-on” device).

The emulsion (E₁) which is a subject of the present invention may be used as cleansing or makeup-removing milk, as cleansing or makeup-removing lotion, as cleansing agent for the face or for the body, as shampoo for cleansing the hair and/or the scalp, as conditioner for treating the hair and/or the scalp, as foam or mousse for caring for or protecting the face, the hands and the body, for example as an antisun protecting agent, as a self-tanning agent, as an antiaging agent, as an antiwrinkle agent, as a calmative or as a moisturizer.

The emulsion (E₁) which is a subject of the present invention may furthermore comprise excipients and/or active agents commonly used in the field of formulations for topical use, in particular cosmetic, dermocosmetic, pharmaceutical or dermopharmaceutical formulations.

The emulsion (E₁) which is a subject of the present invention and as defined above, may also comprise one or more auxiliary compounds selected from foaming and/or detergent surfactants, thickening and/or gelling surfactants, stabilizing agents, film-forming compounds, solvents and cosolvents, hydrotropic agents, plasticizers, opacifiers, nacreous agents, overfatting agents, sequestrants, chelating agents, antioxidants, fragrances, essential oils, preserving agents, conditioning agents, deodorants, bleaching agents intended for bleaching body hair and the skin, active agents intended to provide a treating and/or protective action to the skin or the hair, sunscreens, mineral fillers or pigments, particles that give a visual effect or that are intended for encapsulating active agents, exfoliant particles, texture agents, optical brighteners and insect repellents.

As examples of foaming and/or detergent surfactants optionally present in the emulsion (E₁) which is a subject of the present invention, mention may be made of topically acceptable anionic, cationic, amphoteric or nonionic foaming and/or detergent surfactants commonly used in this sector of activity.

Among the foaming and/or detergent anionic surfactants that may be combined with the emulsion (E₁) which is a subject of the present invention, mention may be made of alkali metal salts, alkaline-earth metal salts, ammonium salts, amine salts, amino alcohol salts of alkyl ether sulfates, of alkyl sulfates, of alkylamido ether sulfates, of alkylarylpolyether sulfates, of monoglyceride sulfates, of α-olefin sulfonates, of paraffin sulfonates, of alkyl phosphates, of alkyl ether phosphates, of alkyl sulfonates, of alkylamide sulfonates, of alkylaryl sulfonates, of alkyl carboxylates, of alkylsulfosuccinates, of alkyl ether sulfosuccinates, of alkylamide sulfosuccinates, of alkyl sulfoacetates, of alkyl sarcosinates, of acylisethionates, of N-acyl taurates, of acyl lactylates, of N-acylamino acid derivatives, of N-acyl peptide derivatives, of N-acyl protein derivatives or of fatty acids.

Among the foaming and/or detergent amphoteric surfactants optionally present in the emulsion (E₁) which is a subject of the present invention, mention may be made of alkylbetaines, alkylamidobetaines, sultaines, alkylamidoalkylsulfobetaines, imidazoline derivatives, phosphobetaines, amphopolyacetates and amphopropionates.

Among the foaming and/or detergent cationic surfactants optionally present in the emulsion (E₁) which is a subject of the present invention, mention may be made particularly of quaternary ammonium derivatives.

Among the foaming and/or detergent nonionic surfactants optionally present in the emulsion (E₁) which is a subject of the present invention, mention may be made more particularly of alkylpolyglycosides comprising a linear or branched, saturated or unsaturated aliphatic radical and comprising from 8 to 12 carbon atoms; castor oil derivatives, polysorbates, coconut amides and N-alkylamines.

As examples of thickening and/or gelling surfactants optionally present in the emulsion (E₁) which is a subject of the present invention, mention may be made of:

optionally alkoxylated alkylpolyglycoside fatty esters, and most particularly ethoxylated methylpolyglucoside esters such as PEG 120 methyl glucose trioleate and PEG 120 methyl glucose dioleate sold, respectively, under the names Glucamate™ LT and Glumate™ DOE120;

alkoxylated fatty esters such as PEG 150 pentaerythrityl tetrastearate sold under the name Crothix™ DS53, or PEG 55 propylene glycol oleate sold under the name Antil™ 141;

fatty-chain polyalkylene glycol carbamates such as PPG 14 laureth isophoryl dicarbamate sold under the name Elfacos™ T211, PPG 14 palmeth 60 hexyl dicarbamate sold under the name Elfacos™ GT2125.

As examples of emulsifying surfactants optionally present in the emulsion (E₁) which is a subject of the present invention, mention may be made of nonionic surfactants, anionic surfactants and cationic surfactants.

As examples of emulsifying nonionic surfactants optionally present in the emulsion (E₁) which is a subject of the present invention, mention may be made of fatty acid esters of sorbitol, for example the products sold under the names Montane™80 and Montane™85 and Montane™60; ethoxylated castor oil and ethoxylated hydrogenated castor oil, for example the product sold under the name Simulsol™ 989; compositions comprising glyceryl stearate and stearic acid poly(ethoxylated) with between 5 mol and 150 mol of ethylene oxide, for example the composition comprising stearic acid (ethoxylated) with 135 mol of ethylene oxide and glyceryl stearate sold under the name Simulsol™ 165; ethoxylated sorbitan esters, for example the products sold under the name Montanox™ mannitan esters; ethoxylated mannitan esters; sucrose esters; methylglucoside esters.

As examples of emulsifying anionic surfactants optionally present in the emulsion (E₁) which is a subject of the present invention, mention may be made of decyl phosphate, cetyl phosphate sold under the name Amphisol™, glyceryl stearate citrate; cetearyl sulfate; the arachidyl/behenyl phosphates and arachidyl/behenyl alcohols composition sold under the name Sensanov™WR; soaps, for example sodium stearate or triethanolammonium stearate, salified N-acylamino acid derivatives, such as, for example, stearoyl glutamate.

As examples of emulsifying cationic surfactants optionally present in the emulsion (E₁) which is a subject of the present invention, mention may be made of amine oxides, quaternium-82 and the surfactants described in patent application WO 96/00719 and mainly those in which the fatty chain comprises at least 16 carbon atoms.

As examples of opacifiers and/or nacreous agents optionally present in the emulsion (E₁) which is a subject of the present invention, mention may be made of sodium palmitate, sodium stearate, sodium hydroxystearate, magnesium palmitate, magnesium stearate, magnesium hydroxystearate, ethylene glycol monostearate, ethylene glycol distearate, polyethylene glycol monostearate, polyethylene glycol distearate and fatty alcohols comprising from 12 to 22 carbon atoms.

As examples of texture agents optionally present in the emulsion (E₁) which is a subject of the present invention, mention may be made of N-acylamino acid derivatives, for example lauroyl lysine sold under the name Aminohope™LL, octenyl starch succinate sold under the name Dryflo™, myristyl polyglucoside sold under the name Montanov™ 14, cellulose fibers, cotton fibers, chitosan fibers, talc, sericite and mica.

As examples of solvents and cosolvents optionally present in the emulsion (E₁) which is a subject of the present invention, mention may be made of water, organic solvents, for example glycerol, diglycerol, glycerol oligomers, ethylene glycol, propylene glycol, butylene glycol, hexylene glycol, diethylene glycol, xylitol, erythritol, sorbitol, water-soluble alcohols such as ethanol, isopropanol or butanol, and mixtures of water and of said organic solvents.

As examples of spring or mineral waters optionally present in the emulsion (E₁) which is a subject of the present invention, mention may be made of spring or mineral waters having a mineralization of at least 300 mg/I, in particular Avene water, Vittel water, Vichy basin water, Uriage water, La Roche Posay water, La Bourboule water, Enghien-les-bains water, Saint-Gervais-les-bains water, Néris-les-bains water, Allevard-les-bains water, Digne water, Maizieres water, Neyrac-les-bains water, Lons le Saunier water, Rochefort water, Saint Christau water, Fumades water and Tercis-les-bains water.

As examples of hydrotropic agents optionally present in the emulsion (E₁) which is a subject of the present invention, mention may be made of xylene sulfonates, cumene sulfonates, hexyl polyglucosides, (2-ethylhexyl) polyglucoside or n-heptyl polyglucoside.

As examples of deodorants optionally present in the emulsion (E₁) which is a subject of the present invention, mention may be made of alkali metal silicates, zinc salts such as zinc sulfate, zinc gluconate, zinc chloride or zinc lactate; quaternary ammonium salts such as cetyltrimethylammonium salts or cetylpyridinium salts; glycerol derivatives such as glyceryl caprate, glyceryl caprylate and polyglyceryl caprate; 1,2-decanediol, 1,3-propanediol; salicylic acid; sodium bicarbonate; cyclodextrins; metallic zeolites; Triclosan™; aluminum bromohydrate, aluminum chlorohydrates, aluminum chloride, aluminum sulfate, aluminum zirconium chlorohydrates, aluminum zirconium trichlorohydrate, aluminum zirconium tetrachlorohydrate, aluminum zirconium pentachlorohydrate, aluminum zirconium octachlorohydrate, aluminum sulfate, sodium aluminum lactate, complexes of aluminum chlorohydrate and of glycol, such as the complex of aluminum chlorohydrate and of propylene glycol, the complex of aluminum dichlorohydrate and of propylene glycol, the complex of aluminum sesquichlorohydrate and of propylene glycol, the complex of aluminum chlorohydrate and of polyethylene glycol, the complex of aluminum dichlorohydrate and of polyethylene glycol, or the complex of aluminum sesquichlorohydrate and of polyethylene glycol.

As examples of active agents optionally present in the emulsion (E₁) which is a subject of the present invention, mention may be made of vitamins and derivatives thereof, especially esters thereof, such as retinol (vitamin A) and the esters thereof (retinyl palmitate, for example), ascorbic acid (vitamin C) and the esters thereof, sugar derivatives of ascorbic acid (such as ascorbyl glucoside), tocopherol (vitamin E) and the esters thereof (such as tocopheryl acetate), vitamin B3 or B10 (niacinamide and derivatives thereof); compounds showing a skin-lightening or skin-depigmenting action, such as the ω-undecelynoyl phenylalanine sold under the name Sepiwhite™MSH, Sepicalm™VG, the glycerol monoester and/or diester of ω-undecelynoyl phenylalanine, ω-undecelynoyl dipeptides, arbutin, kojic acid, hydroquinone; compounds showing a soothing action, especially Sepicalm™ S, allantoin and bisabolol; anti-inflammatories; compounds showing a moisturizing action, such as urea, hydroxyureas, glycerol, polyglycerols, glycerolglucoside, diglycerolglucoside, polyglycerylglucosides, xylitylglucoside; the composition sold under the trade name Aquaxyl™, the composition sold under the trade name Pro-xylane™, derivatives of C-glycosides and more particularly derivatives of C-glucosides and C-xylosides; polyphenol-rich plant extracts, such as grape extracts, pine extracts, wine extracts, olive extracts; compounds showing a slimming or lipolytic action, such as caffeine or derivatives thereof, Adiposlim™, Adipoless™, fucoxanthine; N-acylated proteins; N-acylated peptides, such as Matrixil™; N-acylated amino acids; partial hydrolyzates of N-acylated proteins; amino acids; peptides; total protein hydrolyzates; soybean extracts, for example Raffermine™; wheat extracts, for example Tensine™ or Gliadine™; plant extracts, such as tannin-rich plant extracts, isoflavone-rich plant extracts or terpene-rich plant extracts; extracts of freshwater or seawater algae; marine plant extracts; marine extracts in general, such as corals; essential waxes; bacterial extracts; ceramides; phospholipids; compounds showing an antimicrobial action or a purifying action, such as Lipacide™ C8G, Lipacide™ UG, Sepicontrol™ A5, Fluidipure™8G; Octopirox™ or Sensiva™ SC50; compounds showing an energizing or stimulating property, such as Physiogenyl™, panthenol and derivatives thereof, such as Sepicap™ MP; anti-aging active agents, such as Sepilift™ DPHP, Lipacide™ PVB, Sepivinol™, Sepivital™, Manoliva™, Phyto-Age™, Timecode™; Survicode™; anti-photoaging active agents; agents for protecting the integrity of the dermoepidermal junction; active agents for increasing the synthesis of extracellular matrix components such as collagen, elastins, glycosaminoglycans; active agents which act favorably on chemical cell communication, such as cytokines, or physical cell communication, such as integrins; active agents which create a “heating” sensation on the skin, such as skin microcirculation activators (such as nicotinic acid derivatives) or products which create a feeling of “freshness” on the skin (such as menthol and derivatives); active agents for improving skin microcirculation, for example veinotonics; draining active agents; active agents for decongestive purposes, such as extracts of gingko biloba, of ivy, of horse chestnut, of bamboo, of ruscus, of butcher's broom, of Centalla asiatica, of fucus, of rosemary, of willow; agents for tanning or browning the skin, such as dihydroxyacetone (DHA), erythrulose, mesotartaric aldehyde, glutaraldehyde, glyceraldehyde, alloxane, ninhydrin, plant extracts, for example extracts of redwood of the genus Pterocarpus and of the genus Baphia, such as Pteropcarpus santalinus, Pterocarpus osun, Pterocarpus soyauxii, Pterocarpus erinaceus, Pterocarpus indicus or Baphia nitida, such as those described in European patent application EP 0 971 683; agents known for their action of facilitating and/or accelerating tanning and/or browning of the human skin and/or for their action of coloring the human skin, for example carotenoids (and more particularly beta-carotene and gamma-carotene), the product sold under the trade name “Carrot oil” (INCI name: Daucus Carota, helianthus annuus Sunflower oil) by Provital, which contains carotenoids, vitamin E and vitamin K; tyrosine and/or the derivatives thereof, known for their effect on accelerating tanning of the human skin in combination with exposure to ultraviolet radiation, for example the product sold under the trade name “SunTan Accelerator™” by Provital, which contains tyrosine and riboflavins (vitamin B), the tyrosine and tyrosinase complex sold under the trade name “Zymo Tan Complex” by Zymo Line, the product sold under the trade name MelanoBronze™ (INCI name: Acetyl Tyrosine, Monk's pepper extract (Vitex Agnus-castus) by Mibelle, which contains acetyl tyrosine, the product sold under the trade name Unipertan VEG-24/242/2002 (INCI name: butylene glycol and Acetyl Tyrosine and hydrolyzed vegetable protein and Adenosine triphosphate) by Unipex, the product sold under the trade name “Try-Excell™” (INCI name: Oleoyl Tyrosine and Luffa Cylindrica (Seed Oil and Oleic acid) by Sederma, which contains extracts of marrow seed (or loofah oil), the product sold under the trade name “Actibronze™” (INCI name: hydrolyzed wheat protein and acetyl tyrosine and copper gluconate) by Alban Muller, the product sold under the trade name Tyrostan™ (INCI name: potassium caproyl tyrosine) by Synerga, the product sold under the trade name Tyrosinol (INCI name: Sorbitan Isostearate, glyceryl oleate, caproyl Tyrosine) by Synerga, the product sold under the trade name InstaBronze™ (INCI name: Dihydroxyacetone and acetyl tyrosine and copper gluconate) sold by Alban Muller, the product sold under the trade name Tyrosilane (INCI name: methylsilanol and acetyl tyrosine) by Exymol; peptides known for their melanogenesis-activating effect, for example the product sold under the trade name Bronzing SF Peptide powder (INCI name: Dextran and Octapeptide-5) by Infinitec Activos, the product sold under the trade name Melitane (INCI name: Glycerin and Aqua and Dextran and Acetyl hexapeptide-1) comprising the acetyl hexapeptide-1 known for its alpha-MSH agonist action, the product sold under the trade name Melatimes Solutions™ (INCI name: Butylene glycol, Palmitoyl Tripeptide-40) by Lipotec, sugars and sugar derivatives, for example the product sold under the trade name Tanositol™ (INCI name: inositol) by Provital, the product sold under the trade name Thalitan™ (or Phycosaccharide™ AG) by CODIF international (INCI name: Aqua and hydrolyzed algin (Laminaria Digitata) and magnesium sulfate and manganese sulfate) containing an oligosaccharide of marine origin (guluronic acid and mannuronic acid chelated with magnesium and manganese ions), the product sold under the trade name Melactiva™ (INCI name: Maltodextrin, Mucuna Pruriens Seed extract) by Alban Muller, flavonoid-rich compounds, for example the product sold under the trade name “Biotanning” (INCI name: Hydrolyzed citrus Aurantium dulcis fruit extract) by Silab and known to be rich in lemon flavonoids (of the hesperidin type); agents intended for treating head hair and/or body hair, for example agents for protecting the melanocytes of the hair follicle, intended to protect said melanocytes against cytotoxic agents responsible for the senescence and/or apoptosis of said melanocytes, such as mimetics of dopachrome tautomerase activity, selected from those described in the European patent application published under the number EP 1 515 688 A2, the synthetic SOD mimetic molecules, for example manganese complexes, antioxidant compounds, for example cyclodextrin derivatives, silica-containing compounds derived from ascorbic acid, lysine or arginine pyrrolidone carboxylate, combinations of mono- and diesters of cinnamic acid and of vitamin C, and more generally those mentioned in the European patent application published under the number EP 1 515 688 A2.

As examples of antioxidants optionally present in the emulsion (E₁) which is a subject of the present invention, mention may be made of EDTA and its salts, citric acid, tartaric acid, oxalic acid, BHA (butylhydroxyanisol), BHT (butylhydroxytoluene), tocopherol derivatives such as tocopherol acetate, mixtures of antioxidant compounds such as Dissolvine™ GL 47S sold by Akzo Nobel under the INCI name: Tetrasodium Glutamate Diacetate.

As examples of sunscreens optionally present in the emulsion (E₁) which is a subject of the present invention, mention may be made of all those listed in the modified cosmetics directive 76/768/EEC, annex VII.

Among the organic sunscreens optionally present in the emulsion (E₁) which is a subject of the present invention, mention may be made of the family of benzoic acid derivatives, such as para-aminobenzoic acids (PABAs), especially monoglyceryl esters of PABA, ethyl esters of N,N-propoxy PABA, ethyl esters of N,N-diethoxy PABA, ethyl esters of N,N dimethyl PABA, methyl esters of N,N-dimethyl PABA, butyl esters of N,N-dimethyl PABA; the family of anthranilic acid derivatives, such as homomenthyl-N-acetyl anthranilate; the family of salicylic acid derivatives, such as amyl salicylate, homomenthyl salicylate, ethylhexyl salicylate, phenyl salicylate, benzyl salicylate, or p-isopropanolphenyl salicylate; the family of cinnamic acid derivatives, such as ethylhexyl cinnamate, ethyl-4-isopropyl cinnamate, methyl 2,5-diisopropyl cinnamate, p-methoxypropyl cinnamate, p-methoxyisopropyl cinnamate, p-methoxyisoamyl cinnamate, p-methoxyoctyl cinnamate, (p-methoxy-2-ethylhexyl) cinnamate, (p-methoxy-2-ethoxyethyl) cinnamate, (p-methoxycyclohexyl) cinnamate, ethyl-α-cyano-β-phenyl cinnamate, (2-ethylhexyl)-α-cyano-β-phenyl cinnamate, mono(2-ethylhexanoyl)glyceryl di(paramethoxy) cinnamate; the family of benzophenone derivatives, such as 2,4-dihydroxybenzophenone, 2,2′-dihydroxy-4-methoxybenzophenone, 2,2′,4,4′-tetrahydroxybenzophenone, 2-hydroxy-4-methoxybenzophenone, 2-hydroxy-4-methoxy-4′ methylbenzophenone, 2-hydroxy-4-methoxybenzophenone-5-sulfonate, 4-phenylbenzophenone, 2-ethylhexyl-4′-phenylbenzophenone-2-carboxylate, 2-hydroxy-4-n-octyloxybenzophenone, 4-hydroxy-3-carboxybenzophenone; 3-(4′-methylbenzylidene)-d,I-camphor, 3 (benzylidene)-d,I-camphor, camphor benzalkonium methosulfate; urocanic acid, ethyl urocanate; the family of sulfonic acid derivatives, such as 2-phenylbenzimidazole-5 sulfonic acid and salts thereof; the family of triazine derivatives, such as hydroxyphenyl triazine, ethylhexyloxyhydroxyphenyl-4-methoxyphenyltriazine, 2,4,6-trianillino-(p-carbo-2′ ethylhexyl-1′-oxy)-1,3,5-triazine, benzoic acid 4,4-((6-(((1,1-dimethylethyl)amino)carbonyl)phenyl)amino)-1,3,5-triazine-2,4-diyl diimino) bis(2-ethylhexyl) ester, 2-phenyl-5 methylbenzoxazole, 2,2′-hydroxy-5-methylphenylbenzotriazole, 2-(2′-hydroxy-5′-t-octyl-phenyl)benzotriazole, 2-(2′-hydroxy-5′-methyphenyl)benzotriazole; dibenzazine; dianisoylmethane, 4-methoxy-4″-t-butylbenzoylmethane; 5-(3,3-dimethyl-2-norbornylidene)-3-pentan-2-one; the family of diphenyl acrylate derivatives, such as 2-ethylhexyl-2-cyano-3,3-diphenyl-2-propenoate, ethyl-2-cyano-3,3-diphenyl-2 propenoate; the family of polysiloxanes, such as benzylidene siloxane malonate.

Among the inorganic sunscreens, also called “mineral screens”, optionally present in the emulsion (E₁) which is a subject of the present invention, mention may be made of titanium oxides, zinc oxides, cerium oxide, zirconium oxide, yellow, red or black iron oxides, and chromium oxides. These mineral screens may or may not be micronized, may or may not have undergone surface treatments and may be optionally provided in the form of aqueous or oily predispersions.

Another subject of the invention is a process for preparing an emulsion (E₁) of oil-in-water type in the form of a foam and as defined above, comprising:

at least one step a) of preparing a phase (A′) by mixing the crosslinked anionic polyelectrolyte (P) and optionally the polysaccharide (PS) in the fatty phase (A₂); and

at least one step b) of emulsifying said phase (A′) obtained at the end of step a) with the cosmetically acceptable aqueous phase (A₁) to obtain an emulsion (E′₁); and

at least one step c) of obtaining the emulsion (E₁) by mixing the inorganic thickening agent (Ei) in the emulsion (E′₁) obtained at the end of step b).

Step a) of preparing a phase (A′) by mixing the crosslinked anionic polyelectrolyte (P) and optionally the polysaccharide (PS) in the fatty phase (A₂) is generally carried out at a temperature greater than or equal to 20° C. and less than or equal to 85° C., more particularly greater than or equal to 20° C. and less than or equal to 80° C., and even more particularly greater than or equal to 40° C. and less than or equal to 80° C.; it is carried out under mechanical stirring at a moderate speed of greater than or equal to 50 rpm and less than or equal to 100 rpm.

Step b) of emulsifying said phase (A′) obtained at the end of step a) with the cosmetically acceptable aqueous phase (Aᵢ) to obtain an emulsion (E′₁) is generally carried out at a temperature greater than or equal to 20° C. and less than or equal to 85° C., more particularly greater than or equal to 20° C. and less than or equal to 80° C., and even more particularly greater than or equal to 40° C. and less than or equal to 80° C.; it is carried out under mechanical stirring by means of a mechanical shearing device, such as, for example, a rotor-stator system, at a shearing rate greater than or equal to 1000 rpm and less than or equal to 8000 rpm, more particularly greater than or equal to 2000 rpm and less than or equal to 8000 rpm, and even more particularly greater than or equal to 4000 rpm and less than or equal to 8000 rpm.

The emulsion (E′₁) obtained at the end of carrying out step b) of the process which is a subject of the present invention has a smooth oil-in-water emulsion appearance and does not have dispersed bubbles of gas: the emulsion (E₁) obtained at the end of carrying out said step b) is not in the form of an oil-in-water emulsion in foam form.

The emulsion (E′₁) obtained at the end of carrying out step b) of the process which is a subject of the present invention may optionally be placed under an atmosphere of a gas other than air, by inerting the headspace of the reactor containing the emulsion (E′₁); said inerting consisting of flushing the atmosphere with the gas selected from nitrogen, carbon dioxide, oxygen or argon and resulting in the substitution of the atmosphere of air with an atmosphere of said gas.

Step c) of obtaining the emulsion (E₁) by mixing the inorganic thickening agent (Ei) in the emulsion (E′₁) obtained at the end of step b) is generally carried out at a temperature greater than or equal to 20° C. and less than or equal to 85° C., more particularly greater than or equal to 20° C. and less than or equal to 80° C., and even more particularly greater than or equal to 40° C. and less than or equal to 80° C.; it is carried out under mechanical stirring by means of a mechanical shearing device, such as, for example, a rotor-stator system, at a shearing rate greater than or equal to 1000 rpm and less than or equal to 8000 rpm, more particularly greater than or equal to 2000 rpm and less than or equal to 8000 rpm, and even more particularly greater than or equal to 4000 rpm and less than or equal to 8000 rpm.

The emulsion (E₁) obtained at the end of carrying out step c) of the process that is a subject of the present invention is an oil-in-water emulsion which is in the form of a foam.

The emulsion (E₁) obtained at the end of carrying out step c) of the process that is a subject of the present invention can then be cooled to reach a temperature of less than or equal to 35° C.

Another subject of the invention is the use of the emulsion (E₁) as defined above for the cosmetic treatment of the skin, hair, scalp, mucous membranes and/or nails.

According to a particular aspect, a subject of the invention is the use of the emulsion (E₁) as described above, for cleansing, protecting and/or caring for the skin, hair, scalp, mucous membranes and/or nails.

The use of the emulsion (E₁) for caring for the skin is understood in the context of the present invention to mean any use intended to hydrate the upper layers of the human skin (epidermis and dermis), intended to protect the human skin against UV-A and UV-B radiation from light sources such as the sun, intended to artificially color human skin to make it brown, intended to lighten human skin, intended to reduce or eliminate the unsightly effects associated with the presence of shadows under the eyes or peri-ocular eye bags, intended to reduce or eliminate any inflammation of the human skin, intended to prevent or reduce or eliminate any bacterial or fungal contamination of human skin, and intended to prevent or slow the appearance of the signs of aging of the human skin or else to eliminate said signs; said signs of aging of the human skin or the lips are the lack of elasticity and/or tone of the human skin or the lips, or the lack of density and/or firmness of the human skin or the lips, or alteration of the microrelief of the human skin or of the lips.

According to another particular aspect, a subject of the invention is the use of the emulsion (E₁) as described above for making up the skin, the hair and/or the nails.

The following examples illustrate the invention without, however, limiting it.

### I)—PREPARATION OF A CROSSLINKED ANIONIC POLYELECTROLYTE (P)

### Terpolymer of ammonium 2-methyl-2-[(1-oxo-2-propenyl)amino]1-propanesulfonate, N,N-dimethyl acrylamide and tetraethoxylated lauryl methacrylate [AMPSNH₄/DMAM/MAL (4 EO) 77.4/19.2/3.4 molar], crosslinked with trimethylolpropanetriacrylate (TMPTA).

592 g of an aqueous solution containing 15% by weight of ammonium 2-methyl-2-[(1-oxo-2-propenyl)amino]1-propanesulfonate (AMPSNH₄) in a tert-butanol/water mixture (97.5/2.5 by volume), 10.1 g of N,N-dimethyl acrylamide (DMAM), 4.2 g of tetraethoxylated lauryl methacrylate [MAL (4 EO)] and 0.75 g of TMPTA are loaded into a reactor maintained at 25° C. with stirring. After a sufficient time to achieve good homogenization of the solution, it is deoxygenated by bubbling with nitrogen heated to 70° C. 0.42 g of dilauroyl peroxide are then added and the reaction medium is then maintained for approximately 60 minutes at 70° C. and then for 2 hours at 80° C. After cooling, the powder which has formed during polymerization is filtered and dried to obtain the desired product, hereinafter referred to as: “Polyelectrolyte (P)”.

### II)—PREPARATION AND EVALUATION OF OIL-IN-WATER EMULSIONS IN THE FORM OF A FOAM ACCORDING TO THE INVENTION AND COMPARATIVE OIL-IN-WATER EMULSIONS

### II-1 Preparation of the Oil-in-Water Emulsions in Foam Form (According to the Invention) and of Comparative Oil-in-Water Emulsions

10 oil-in-water emulsions according to the invention are prepared, denoted (F1) to (F10), the proportions by weight of the constituents of which are indicated in table 1 comparative oil-in-water emulsions denoted (F′1) to (F′7), the proportions by weight of the constituents of which are indicated in table 2 below. The common preparation process for the oil-in-water emulsions (F1) to (F10) and for the oil-in-water emulsions (F′1) to (F′4) is as follows:

the various constituents of the fatty phase are poured into a beaker at a temperature of 85° C. with mechanical stirring at a speed of eighty revolutions per minute,

the polyelectrolyte (P) (or, depending on the case, Sepiplus™ 400 or Pemulen™ TR2) and Solagum™ AX are gradually and successively dispersed in the beaker containing the fatty phase with mechanical stirring at a speed of eighty revolutions per minute;

the various constituents of the aqueous phase are poured into another beaker at a temperature of 85° C. with mechanical stirring at a speed of eighty revolutions per minute;

the content of the beaker comprising the aqueous phase is gradually added to the fatty phase comprising the polymer system at a temperature of 85° C. with mechanical stirring by means of a stirring rotor equipped with a rotor-stator system at four thousand revolutions per minute for four minutes;

the organic thickener (kaolin or red clay or green clay), previously placed in the form of an aqueous dispersion, is then introduced into the previously formed mixture at a temperature of 85° C.;

the mixture thus obtained is then subjected to shearing mechanical stirring by means of a stirring rotor equipped with a rotor-stator system at four thousand revolutions per minute for four minutes; then cooled to a temperature of 25° C., then emptied out to obtain the emulsions to be evaluated.

The process for preparing the comparative oil-in-water emulsion (F′5) is as follows:

the various constituents of the fatty phase are poured into a beaker at a temperature of 85° C. with mechanical stirring at a speed of eighty revolutions per minute,

the polyelectrolyte (P) and the Solagum™ AX are gradually and successively dispersed in the beaker containing the fatty phase with mechanical stirring at a speed of eighty revolutions per minute;

the various constituents of the aqueous phase are poured into another beaker at a temperature of 85° C. with mechanical stirring at a speed of eighty revolutions per minute;

the content of the beaker comprising the aqueous phase is gradually added to the fatty phase comprising the polymer system at a temperature of 85° C. with mechanical stirring by means of a stirring rotor equipped with a rotor-stator system at four thousand revolutions per minute;

the mixture thus obtained is cooled to 20° C., then emptied out to obtain the emulsion (F′5) to be evaluated.

The process for preparing the comparative oil-in-water emulsion (F′6) comprises all the steps used in the process for preparing the comparative oil-in-water emulsion (F′5), with the exception of the emptying out step, followed by an additional step of introducing nitrogen at a temperature of 20° C. for a period of thirty minutes by means of a suitable gas inlet device. The mixture thus obtained is cooled to 20° C., then emptied out to obtain the emulsion (F′6) to be evaluated.

The process for preparing the comparative oil-in-water emulsion (F′7) comprises all the steps used in the process for preparing the comparative oil-in-water emulsion (F′5), with the exception of the emptying out step, followed by an additional step of introducing nitrogen at a temperature of 60° C. for a period of 30 minutes by means of a suitable gas inlet device. The mixture thus obtained is cooled, still with stirring and under nitrogen bubbling, to reach a temperature of 20° C., then the introduction of nitrogen and the stirring are stopped in order to empty out and to obtain the emulsion (F′7) to be evaluated.

TABLE 1

| | Emulsion | | | | | |
|---|---|---|---|---|---|---|
| | (F1) | (F2) | (F3) | (F4) | (F5) | (F6) |
| Fatty phase | | | | | | |
| Glyceryl stearate | 2% | 2% | 2% | 2% | 2% | 2% |
| Neopentyl glycol | 6% | 6% | 6% | 6% | 6% | 6% |
| diethylhexanoate | | | | | | |
| Isononyl isononanoate | 6% | 6% | 6% | 6% | 6% | 6% |
| Emulsifying system | | | | | | |
| Montanov 202⁽¹⁾ | 2% | 2% | 2% | 2% | 2% | 2% |
| Montanov 68⁽²⁾ | 1% | 1% | 1% | 1% | 1% | 1% |
| Polymer system | | | | | | |
| Polyelectrolyte (P) | 0.5% | 1% | 1.5% | 1% | 1% | 1% |
| Solagum AX⁽³⁾ | 0.2% | 0.2% | 0.2% | 0.2% | 0.2% | 0.2% |
| Inorganic thickener (Ei) | | | | | | |
| Kaolin | 10% | 10% | 10% | 8% | 12% | 15% |
| Green clay | 0% | 0% | 0% | 0% | 0% | 0% |
| Red clay | 0% | 0% | 0% | 0% | 0% | 0% |
| Aqueous phase | | | | | | |
| Water | Qs 100% | Qs 100% | Qs 100% | Qs 100% | Qs 100% | Qs 100% |
| Euxyl PE 9010⁽⁴⁾ | 1% | 1% | 1% | 1% | 1% | 1% |

Emulsion

| | (F7) | (F8) | (F9) | (F10) |
|---|---|---|---|---|
| Fatty phase | | | | |
| Glyceryl stearate | 2% | 2% | 2% | 2% |
| Neopentyl glycol | 6% | 6% | 6% | 6% |
| diethylhexanoate | | | | |
| Isononyl isononanoate | 6% | 6% | 6% | 6% |
| Emulsifying system | | | | |
| Montanov 202⁽¹⁾ | 2% | 2% | 0% | 0% |
| Montanov 68⁽²⁾ | 1% | 1% | 0% | 0% |
| Brij ™ S2⁽⁹⁾ | 0% | 0% | 1.5% | 0% |
| Brij ™ S721⁽¹⁰⁾ | 0% | 0% | 1.5% | 0% |
| Alcohols C16/C18 | 0% | 0% | 1% | 1% |
| (50/50)⁽⁵⁾ | | | | |
| Simulsol ™ 165⁽⁶⁾ | 0% | 0% | 0% | 3% |
| Polymer system | | | | |
| Polyelectrolyte (P) | 1% | 1% | 1% | 1% |
| Solagum AX⁽³⁾ | 0.2% | 0.2% | 0.2% | 0.2% |
| Inorganic thickener (Ei) | | | | |
| Kaolin | 0% | 0% | 10% | 10% |
| Green clay | 10% | 0% | 0% | 0% |
| Red clay | 0% | 10% | 0% | 0% |
| Aqueous phase | | | | |
| Water | Qs 100% | Qs 100% | Qs 100% | Qs 100% |
| Euxyl PE 9010⁽⁴⁾ | 1% | 1% | 1% | 1% |

TABLE 2

| | Emulsion | | | | | | |
|---|---|---|---|---|---|---|---|
| | (F′1) | (F′2) | (F′3) | (F′4) | (F′5) | (F′6) | (F′7) |
| Fatty phase | | | | | | | |
| Glyceryl stearate | 2% | 2% | 2% | 2% | 2% | 2% | 2% |
| Neopentyl glycol | 6% | 6% | 6% | 6% | 6% | 6% | 6% |
| diethylhexanoate | | | | | | | |
| Isononyl | 6% | 6% | 6% | 6% | 6% | 6% | 6% |
| isononanoate | | | | | | | |
| Emulsifying system | | | | | | | |
| Montanov 202⁽¹⁾ | 2% | 2% | 2% | 2% | 2% | 2% | 2% |
| Montanov 68⁽²⁾ | 1% | 1% | 1% | 1% | 1% | 1% | 1% |
| Polymer system | | | | | | | |
| Polyelectrolyte (P) | 1% | 0% | 0% | 1% | 1% | 1% | 1% |
| Solagum AX⁽³⁾ | 0.2% | 0.2% | 0.2% | 0.2% | 0.2% | 0.2% | 0.2% |
| Sepiplus 400⁽⁷⁾ | 0% | 1.7% | 0% | 0% | 0% | 0% | 0% |
| Pemulen TR2⁽⁸⁾ | 0% | 0% | 1% | 0.5% | 0% | 0% | 0% |
| Inorganic thickener | | | | | | | |
| (Ei) | | | | | | | |
| Kaolin | 5% | 10% | 10% | 10% | 0% | 0% | 0% |
| Aqueous phase | | | | | | | |
| Water | Qs 100% | Qs 100% | Qs 100% | Qs 100% | Qs 100% | Qs 100% | Qs 100% |
| Euxyl PE 9010⁽⁴⁾ | 1% | 1% | 1% | 1% | 1% | 1% | 1% |

(1) Montanov™ 202 (INCI name: Arachidyl Alcohol & Behenyl Alcohol & Arachidyl Glucoside): Emulsifier sold by SEPPIC;
(2) Montanov™ 68 (INCI name: Cetearyl Alcohol & Cetearyl Glucoside): Emulsifier sold by SEPPIC;
(3) Solagum™ AX (INCI name Acacia Senegal Gum & Xanthan Gum): Thickening agent sold by SEPPIC;
(4) Euxyl™ PE 9010 (INCI name: phenoxyethanol & ethylhexylglycerin): Preserving agent sold by Schülke;
(5) Alcohols C16/C18 (50/50): Mixture of 1-hexadecanol and 1-octyldecanol, in a weight ratio of 50/50;
(6) Simulsol™ 165 (INCI name: PEG-100 stearate & Glyceryl stearate): Emulsifying agent sold by SEPPIC, comprising, per 100% of its weight, a proportion by weight of 50% of PEG-100 stearate and a proportion by weight of 50% of glyceryl stearate;
l (7) Sepiplus™ 400 (INCI name: Polyacrylate 13 & Polyisobutene & Polysorbate 20): Polymeric thickener in the form of a reverse latex comprising a crosslinked polymer comprising acrylamide, sodium 2-acrylamido-2-methylpropanesulfonate, acrylic acid and sodium acrylate, the content by weight of said crosslinked polymer in the reverse latex is equal to 60%;
(8) Pemulen™ TR2 (INCI Name: Acrylates/C10-30 Alkyl Acrylate Crosspolymer): High molecular weight polymeric emulsifier, crosslinked copolymer of acrylic acid and of a hydrophobic comonomer;
(9) Brij™ S2 (INCI name: steareth-2): Emulsifying agent originating from the ethoxylation of stearyl alcohol with 2 mol of ethylene oxide, sold by Croda;
(10) Brij™ S721 (INCI name: steareth-21): Emulsifying agent originating from the ethoxylation of stearyl alcohol with 21 mol of ethylene oxide, sold by Croda.

### II-2 Demonstration of the Properties of the Water-in-Oil Emulsions (F1) to (F10) According to the Invention and the Comparative Water-in-Oil Emulsions (F′1) to (F′7).

II2.1 Characterization of the Appearance and Viscosity of the Water-in-Oil Emulsions (F1) to (F10) According to the Invention and of the Comparative Water-in-Oil Emulsions (F′1) to (F′7).

The emulsions (F1) to (F10) according to the invention and the comparative water-in-oil emulsions (F′1) to (F′7) obtained according to the processes described above are then stored in an insulated air-conditioned chamber regulated at a temperature of 20° C. for one month. After a duration of one day (J1), 7 days (J7) and one month (M1), the appearance (ASP) of each prepared emulsion is observed. After a duration of 7 days (J7) and one month (M1), the dynamic viscosity (μ) of each emulsion is measured (in mPa·s) using a viscometer at 20° C. (Brookfield RVT, spindle 7, five revolutions per minute).

### Expressing the Results:

when the emulsion observed is in the form of a homogeneous emulsion in foam form, the appearance (ASP) is denoted “HMo”;

when the emulsion observed is in the form of a homogeneous emulsion but is not in foam form, the appearance (ASP) is denoted “HNMo”;

when the emulsion observed is in the form of a heterogeneous emulsion due to the presence of lumps and is not in the form of a foam, the appearance (ASP) is denoted “Het-NMo”.

II-2.2 Results Obtained for the Water-in-Oil Emulsions (F1) to (F10) According to the Invention and the Comparative Water-in-Oil emulsions (F′1) to (F′7).

The evaluation methods described in paragraph II-2.1 were applied to the water-in-oil emulsions (F1) to (F10) according to the invention and the comparative water-in-oil emulsions (F′1) to (F′7). The results obtained are recorded in tables 3 and 4 below, respectively for the water-in-oil emulsions (F1) to (F10) according to the invention and the comparative water-in-oil emulsions (F′1) to (F′7).

TABLE 3

| | (F1) | (F2) | (F3) | (F4) | (F5) | (F6) |
|---|---|---|---|---|---|---|
| ASP | HMo | HMo | HMo | HMo | HMo | HMo |
| (J1, 20° C.) | | | | | | |
| ASP | HMo | HMo | HMo | HMo | HMo | HMo |
| (J7, 20° C.) | | | | | | |
| μ (J7) | 41 100 | 124 000 | 264 000 | 88 000 | 149 000 | 210 000 |
| ASP (M1) | HMo | HMo | HMo | HMo | HMo | HMo |
| μ (M1) | 41 200 | 118 000 | 238 000 | 108 000 | 179 000 | 204 000 |
| ASP | HMo | HMo | HMo | HMo | HMo | HMo |
| (J7, 45° C.) | | | | | | |
| ASP | HMo | HMo | HMo | HMo | HMo | HMo |
| (M1, 45° C.) | | | | | | |

| | (F7) | (F8) | (F9) | (F10) |
|---|---|---|---|---|
| ASP (J1, 20° C.) | HMo | HMo | HMo | HMo |
| ASP (J7, 20° C.) | HMo | HMo | HMo | HMo |
| μ (J7) | 160 000 | 240 000 | 135 000 | 161 000 |
| ASP (M1, | HMo | HMo | HMo | HMo |
| 20° C.) | | | | |
| μ (M1) | 171 000 | 156 000 | 122 000 | 160 000 |
| ASP (J7, 45° C.) | HMo | HMo | HMo | HMo |
| ASP (M1, | HMo | HMo | HMo | HMo |
| 45° C.) | | | | |

TABLE 4

| | (F′1) | (F′2) | (F′3) | (F′4) | (F′5) | (F′6) | (F′7) |
|---|---|---|---|---|---|---|---|
| ASP (J1, 20° C.) | HNMo | Het-NMo | Het- | Het- | HNMo | HNMo | HNMo |
| | | | NMo | NMo | | | |
| ASP (J7, 20° C.) | HNMo | Het-NMo | Het- | Het- | HNMo | HNMo | HNMo |
| | | | NMo | NMo | | | |
| μ (J7) | 98 000 | 98 000 | nm | 152 000 | 66 000 | nm | nm |
| ASP (M1, 20° C.) | HNMo | Het-NMo | Het- | Het- | HNMo | HNMo | HNMo |
| | | | NMo | NMo | | | |
| μ (M1) | 102 000 | 97 000 | nm | 220 000 | nm | nm | nm |
| ASP (J7, 45° C.) | HNMo | Het-NMo | Het- | Het- | nm | nm | nm |
| | | | NMo | NMo | | | |
| ASP (M1, 45° C.) | HNMo | Het-NMo | Het- | Het- | nm | nm | nm |
| | | | NMo | NMo | | | |

### II-2.3 Analysis of the Results

The water-in-oil emulsions (F₁), (F₂), (F₃), (F₄), (F₅), (F₆), (F₇), (F₈), (F₉) and (F₁₀) according to the invention are characterized:

by a homogeneous appearance and a foam form after a duration of one day, 7 days and one month of storage at a temperature of 20° C.,

by a homogeneous appearance and a foam form after a duration of 7 days and one month of storage at a temperature of 45° C.,

by satisfactory stability of the dynamic viscosity values measured after 1 month of storage at 20° C., compared with the dynamic viscosity values measured after 7 days of storage at 20° C., said dynamic viscosity values being measured by means of a Brookfield RV model viscometer at 20° C. at a speed of five revolutions per minute.

The comparative emulsion (F′₁), being distinguished from the emulsions (F2), (F4), (F6) and (F7) according to the invention by an (Ei)/(P) weight ratio value equal to 5/1 ((F2), (F4), (F6) and (F7) being characterized by (Ei)/(P) weight ratio values, respectively, of 10/1, 8/1, 12/1 and 15/1), exhibits a heterogeneous appearance and a lack of foam form, whereas the emulsions (F2), (F4), (F6) and (F7) according to the invention exhibit a homogeneous appearance and a foam form.

The comparative emulsion (F′₂) comprising, as polymer system, a crosslinked anionic polyelectrolyte, and being characterized by a weight ratio [inorganic thickener (Ei)/crosslinked anionic polyelectrolyte] equal to 10/1, has a heterogeneous appearance (presence of lumps) and is not in the form of a foam.

The comparative emulsions (F′₃) and (F′₄) comprising, as polymer system, a crosslinked anionic polyelectrolyte, the polymer backbone of which comprises a monomer having a non-ethoxylated alkyl chain and being characterized by weight ratios [inorganic thickener (Ei)/crosslinked anionic polyelectrolyte], respectively equal to 10/1 and 20/1, have a heterogeneous appearance (presence of lumps) and are not in the form of a foam.

The comparative emulsion (F′₅) comprising, as polymer system, the crosslinked anionic polyelectrolyte (P) and not comprising an inorganic thickener (Ei), exhibits a homogeneous appearance and an absence of foam form, whereas the emulsions (F2), (F4), (F6) and (F7) according to the invention exhibit a homogeneous appearance and a foam form.

The comparative emulsions (F′₆) and (F′₇), comprising, as polymer system, the crosslinked anionic polyelectrolyte (P) and not comprising an inorganic thickener (Ei), and the preparation process of which comprises a step of nitrogen bubbling, exhibits a homogeneous appearance and an absence of foam form, whereas the emulsions (F2), (F4), (F6) and (F7) according to the invention exhibit a homogeneous appearance and a foam form.

1. An emulsion (E₁) of oil-in-water type in the form of a foam, comprising, per 100% of its weight:
a)—from 27% to 89.9% by weight of a cosmetically acceptable aqueous phase (A₁);
b)—from 0.1% to 3% by weight of a crosslinked anionic polyelectrolyte (P) resulting from the polymerization, in the presence of at least one crosslinking agent, of partially or totally salified 2-methyl-2-[(1-oxo-2-propenyl)amino]-1-propanesulfonic acid with at least one neutral monomer selected from acrylamide, (2-hydroxyethyl) acrylate, N,N-dialkylacrylamides, in which each of the alkyl groups comprises between one and four carbon atoms, and at least one monomer of formula (I): 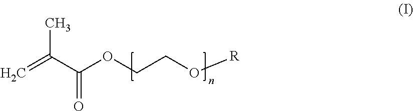
in which R represents a linear or branched alkyl radical comprising from eight to twenty carbon atoms and n represents an integer greater than or equal to one and less than or equal to twenty;
c)—from 5% to 20% by weight of at least one inorganic thickening agent (Ei) chosen from natural or synthetic, modified or unmodified phyllosilicates;
d)—from 5% to 50% by weight of a fatty phase (A₂) comprising, per 100% of its weight:
d₁)—from 0.2% to 25% by weight of an emulsifying system (S) comprising one or more emulsifying surfactants selected from alkylpolyglycoside compositions; alkylpolyglycoside and fatty alcohol compositions; polyethoxylated fatty acids comprising from 10 to 40 carbon atoms and having a degree of ethoxylation of between 5 and 100; compositions of polyethoxylated fatty acids comprising from 10 to 40 carbon atoms and having a degree of ethoxylation of between 5 and 100 and glycerol esters of fatty acids comprising from 10 to 40 carbon atoms; polyethoxylated fatty alcohols comprising from 10 to 40 carbon atoms and having a degree of ethoxylation of between 2 and 100; compositions of polyethoxylated fatty alcohols comprising from 10 to 40 carbon atoms and having a degree of ethoxylation of between 2 and 100 and fatty alcohols comprising from 12 to 40 carbon atoms; compositions of polyethoxylated fatty acids comprising from 10 to 40 carbon atoms and having a degree of ethoxylation of between 5 and 100 and glycerol esters of fatty acids comprising from 10 to 40 carbon atoms and fatty alcohols comprising from 12 to 40 carbon atoms; and
d₂)—from 75% to 99.8% by weight of at least one oil and optionally at least one wax;
said emulsion (E₁) being further characterized in that the weight ratio between the inorganic thickening agent (Ei) and the crosslinked anionic polyelectrolyte (P) is greater than or equal to 6/1 and less than or equal to 20/1.

2. The emulsion (E₁) as defined in claim 1, further comprising, per 100% of its weight:
d)—from 5% to 50% by weight of a fatty phase (A₃) comprising, per 100% of its weight:
d₁)—from 0.2% to 20% by weight of said emulsifying system (S); and
d₂)—from 80% to 99.8% by weight of at least one oil and optionally at least one wax.

3. The emulsion (E₁) as defined in claim 1, further comprising, per 100% of its weight:
a)—from 27% to 84.5% by weight of said aqueous phase (A₁);
b)—from 0.5% to 3% by weight of said crosslinked anionic polyelectrolyte (P).

4. The emulsion (E₁) as defined in claim 1, wherein said crosslinked anionic polyelectrolyte (P) comprises, per 100 mol % of its constituent monomers:
from 20 mol % to 80 mol % of monomer units derived from said partially or totally salified 2-methyl-2-[(1-oxo-2-propenyl)amino]-1-propanesulfonic acid;
from 15 mol % to 75 mol % of monomer units derived from at least one neutral monomer chosen from acrylamide, (2-hydroxyethyl) acrylate, N,N-dialkylacrylamides, in which each of the alkyl groups comprises between one and four carbon atoms,
from 0.5 mol % to 5 mol % of monomer units derived from said monomer of formula (I) as defined above.

5. The emulsion (E₁) as defined in claim 1, wherein said neutral monomer is selected from acrylamide, (2-hydroxyethyl) acrylate or N,N-dimethylacrylamide.

6. The emulsion (E₁) as defined in claim 1, wherein said monomer of formula (I) is tetraethoxylated lauryl methacrylate.

7. The emulsion (E₁) as defined in claim 6, wherein said crosslinked anionic polyelectrolyte (P) is a terpolymer of 2-methyl-2-[(1-oxo-2-propenyl)amino]-1-propanesulfonic acid, partially salified in ammonium salt form, of N,N-dimethylacrylamide and of tetraethoxylated lauryl methacrylate, crosslinked with trimethylolpropane triacrylate.

8. The emulsion (E₁) as defined in claim 1, wherein said crosslinked anionic polyelectrolyte (P) comprises, per 100 mol%:
from 60 mol % to 80 mol % of monomer units derived from 2-methyl-2-[(1-oxo-2-propenyl)amino]-1-propanesulfonic acid, partially salified in ammonium form,
from 15 mol % to 39.5 mol % of monomer units derived from N,N-dimethylacrylamide, and
from 0.5 mol % to 5 mol % of monomer units derived from tetraethoxylated lauryl methacrylate.

9. The emulsion (E₁) as defined in claim 1, wherein the inorganic thickener (Ei) is chosen from kaolinite, montmorillonite, saponite, bentonite or hectorite.

10. The emulsion (E₁) as defined in claim 1, wherein the weight ratio between said inorganic thickening agent (Ei) and said crosslinked anionic polyelectrolyte (P) is greater than or equal to 8/1.

11. The emulsion (E₁) as defined in claim 1, further comprising, per 100% of its weight,
(e)—a proportion by weight greater than or equal to 0.01% and less than or equal to 0.5% of at least one polysaccharide (PS) selected from polysaccharides consisting of monosaccharide derivatives and polysaccharides consisting solely of monosaccharides.

12. The emulsion (E₁) as defined in claim 11, wherein the polysaccharide (PS) is selected from the group consisting of carrageenans, agar, exudate of gum arabic, exudate of gum karaya, xanthan gum, gellan gum, chitosan, glucoans, galactomannans originating from cassia gum, galactomannans originating from locust bean gum, galactomannans originating from tara gum, galactomannans originating from guar gum and galactomannans originating from fenugreek gum.

13. The emulsion (E₁) as defined in claim 11, further comprising, per 100% of its weight:
a)—from 52.5% to 76.4% by weight of a cosmetically acceptable aqueous phase (A₁);
b)—from 0.5% to 2% by weight of a crosslinked anionic polyelectrolyte (P) resulting from the polymerization, in the presence of at least one crosslinking agent, of from 60 mol % to 80 mol % of monomer units derived from 2-methyl-2-[(1-oxo-2-propenyl)amino]-1-propanesulfonic acid, partially or totally salified in ammonium salt form, with from 15 mol % to 39.5 mol % of monomer units derived from N,N-dimethylacrylamide, and from 0.5 mol % to 5 mol % of monomer units derived from tetraethoxylated lauryl methacrylate,
c)—from 8% to 15% by weight of at least one inorganic thickening agent (Ei) selected from among kaolinite, montmorillonite, saponite, bentonite or hectorite,
d)—from 15% to 30% by weight of a fatty phase (A₂) comprising, per 100% of its weight:
d₁)—from 5% to 25% by weight of an emulsifying system (S) comprising one or more emulsifying surfactants selected from alkylpolyglycoside compositions; alkylpolyglycoside and fatty alcohol compositions; polyethoxylated fatty acids comprising from 10 to 40 carbon atoms and having a degree of ethoxylation of between 5 and 100; compositions of polyethoxylated fatty acids comprising from 10 to 40 carbon atoms and having a degree of ethoxylation of between 5 and 100 and glycerol esters of fatty acids comprising from 10 to 40 carbon atoms; polyethoxylated fatty alcohols comprising from 10 to 40 carbon atoms and having a degree of ethoxylation of between 2 and 100; compositions of polyethoxylated fatty alcohols comprising from 10 to 40 carbon atoms and having a degree of ethoxylation of between 2 and 100 and fatty alcohols comprising from 12 to 40 carbon atoms; compositions of polyethoxylated fatty acids comprising from 10 to 40 carbon atoms and having a degree of ethoxylation of between 5 and 100 and glycerol esters of fatty acids comprising from 10 to 40 carbon atoms and fatty alcohols comprising from 12 to 40 carbon atoms; and
d₂)—from 75% to 95% by weight of at least one oil and optionally at least one wax;
e)—from 0.1% to 0.5% by weight of at least one polysaccharide selected from agar, exudate of gum arabic, exudate of gum karaya, xanthan gum, gellan gum, galactomannans originating from cassia gum, galactomannans originating from locust bean gum, galactomannans originating from tara gum, galactomannans originating from guar gum and galactomannans originating from fenugreek gum,
wherein in said emulsion (E₁), the weight ratio between the inorganic thickening agent (Ei) and the crosslinked anionic polyelectrolyte (P) is greater than or equal to 8/1 and less than or equal to 20/1.

14. A process for preparing an emulsion (E₁) of oil-in-water type in the form of a foam and as defined in claim 1, comprising:
at least one step a) of preparing a phase (A′) by mixing the crosslinked anionic polyelectrolyte (P) and optionally the polysaccharide (PS) in the fatty phase (A₂); and
at least one step b) of emulsifying said phase (A′) obtained at the end of step a) with the cosmetically acceptable aqueous phase (A₁) to obtain an emulsion (E′₁); and
at least one step c) of obtaining the emulsion (E₁) by mixing the inorganic thickening agent (Ei) in the emulsion (E′₁) obtained at the end of step b).

15. A method for cosmetic treatment of the skin, hair, scalp, mucous membranes and/or nails, comprising providing the emulsion (E₁) of claim 1, and applying an effective amount of the emulsion.

16. A method for cleansing, protecting and/or caring for the skin, hair, scalp, mucous membranes and/or nails, and for making up the skin, hair and/or nails, comprising providing the emulsion (E₁) of claim 1, and applying an effective amount of the emulsion.

17. The emulsion (E₁) as defined in claim 2, wherein said crosslinked anionic polyelectrolyte (P) comprises, per 100 mol % of its constituent monomers:
from 20 mol % to 80 mol % of monomer units derived from said partially or totally salified 2-methyl-2-[(1-oxo-2-propenyl)amino]-1-propanesulfonic acid;
from 15 mol % to 75 mol % of monomer units derived from at least one neutral monomer chosen from acrylamide, (2-hydroxyethyl) acrylate, N,N-dialkylacrylamides, in which each of the alkyl groups comprises between one and four carbon atoms,
from 0.5 mol % to 5 mol % of monomer units derived from said monomer of formula (I) as defined above.

18. The emulsion (E₁) as defined in claim 3, wherein said crosslinked anionic polyelectrolyte (P) comprises, per 100 mol % of its constituent monomers:
from 20 mol % to 80 mol % of monomer units derived from said partially or totally salified 2-methyl-2-[(1-oxo-2-propenyl)amino]-1-propanesulfonic acid;
from 15 mol % to 75 mol % of monomer units derived from at least one neutral monomer chosen from acrylamide, (2-hydroxyethyl) acrylate, N,N-dialkylacrylamides, in which each of the alkyl groups comprises between one and four carbon atoms,
from 0.5 mol % to 5 mol % of monomer units derived from said monomer of formula (I) as defined above.

19. The emulsion (E₁) as defined in claim 2, wherein said neutral monomer is selected from acrylamide, (2-hydroxyethyl) acrylate or N,N-dimethylacrylamide.

20. The emulsion (E₁) as defined in claim 3, wherein said neutral monomer is selected from acrylamide, (2-hydroxyethyl) acrylate or N,N-dimethylacrylamide.
